(19) United States
(12) Patent Application Publication
(10) Pub. No.: US 2024/0263129 A1
(43) Pub. Date: Aug. 8, 2024

(54) METHODS FOR THE PRODUCTION OF MYCELIAL BIOMASS FROM DATE EXTRACT

(71) Applicant: MYCOTECHNOLOGY, INC., Aurora, CO (US)

(72) Inventors: Berenice VERGARA-PORRAS, Aurora, CO (US); Phillip VO, Aurora, CO (US); Bassam ALKOTAINI, Aurora, CO (US); James Patrick LANGAN, Aurora, CO (US); Miles INGWERS, Aurora, CO (US); Matthew MALONE, Aurora, CO (US); Ross CORNELISSEN, Aurora, CO (US); Rick Lynn BECKER, Aurora, CO (US); Megan McMASTER, Aurora, CO (US); Brendan SHARKEY, Aurora, CO (US); Chase GARDNER, Aurora, CO (US); Joseph MEILEN, Aurora, CO (US); Nicholas DAWSON, Aurora, CO (US); Todd McDONALD, Aurora, CO (US); Anthony J. CLARK, Aurora, CO (US); Marina NADAL, Aurora, CO (US)

(73) Assignee: MYCOTECHNOLOGY, INC., Aurora, CO (US)

(21) Appl. No.: 18/566,975

(22) PCT Filed: Jun. 10, 2022
(86) PCT No.: PCT/US2022/032997

§ 371 Date: Dec. 4, 2023

Related U.S. Application Data
(60) Provisional application No. 63/209,843, filed on Jun. 11, 2021.

(51) Int. Cl. C12N 1/14; A23L 19/00

(52) U.S. Cl. CPC C12N 1/14; A23L 19/00; C12N 2500/34; C12N 2500/46; C12N 2500/76

(57) ABSTRACT

Provided are methods to produce an edible filamentous fungal biomass using an aqueous media which has a carbon source including an extract of dates; and a nitrogen source into which is inoculated filamentous fungal culture followed by culturing in a submerged fungal culture to produce an edible filamentous fungal biomass, wherein the fungal culture comprises Pleurotus spp. The culture may be grown to at least about 25 g/L (dry weight) with a productivity of at least 2.5 g/L/day (dry weight) during the culturing step. Also provided herein are compositions including an edible filamentous fungus.

### CROSS REFERENCE TO RELATED APPLICATIONS

This patent application claims the benefit of U.S. Provisional Patent Application No. 63/209,843, filed Jun. 11, 2021, which is incorporated by reference in its entirety.

### BACKGROUND OF THE INVENTION

The date palm Phoenix dactylifera, a tropical and subtropical tree, belonging to the family Palmae (Arecaceae) is one of mankind's oldest cultivated plants. Today, the production of date fruits is on the increase as recorded for some of the major date producing countries like Oman, Egypt, Saudi Arabia, and the UAE. P. dactylifera is the primary crop in Oman.

Whole date fruits are traditionally used to produce a wide range of products such as date juice concentrates (spread, syrup and liquid sugar), fermented date products (wine, alcohol, vinegar, and organic acids) and date pastes for different uses (e.g. bakery and confectionary) besides their direct consumption. Date pectin, dietary fiber and syrup are some of the date substances which find applications as a thickener or gelling agent in processed foods, i.e. confectionery products, jams, table jellies, soft cheeses, yoghurts, etc.

Date syrup (dibs), the main and general by-product of date, is used in the preparation of foodstuffs such as jams, marmalades, concentrated beverages, chocolates, ice cream, confectioneries, sweets, snacks, bakery products and health. In the date syrup industry, the fruits are mixed with water and heated for around 1 h at 50° C. and the main component, sugars, are then extracted. It is also produced in homes and in villages by extraction and boiling down of juice and on a semi and full industrial scale (FAO, 1992). Mature date fruits are also processed into products such as date bars, date syrup, etc.

Dates are rich in sugar ranging from 65% to 80% on dry weight basis mostly of monosaccarides (primarily, glucose and fructose). Fresh varieties have a higher content of monosaccharides. Water content is between 7% (dried) and 79% (fresh) depending on variety. Date syrup is nutritious: it contains 740 mg potassium, where honey has 52 mg and maple syrup, 212 mg. For iron, date syrup contains 0.44 mg as compared to 0.42 mg in honey and 0.11 in maple syrup. For magnesium, date syrup contains 46 mg, whereas honey only contains 2 mg and maple syrup, 21 mg. Finally, for phosphorus, date syrup contains 50 mg where honey contains 4 mg and maple syrup, 2 mg.

Per a serving size of 1 tablespoon, date syrup provides 17 g of carbohydrates, with 13 g of that being sugar, and no protein or fat. On the other hand, USDA Dietary Guidelines recommends consuming 10-35% of calories from protein, 45-65% from carbohydrates, and 20-35% of calories from fat. It would be desirable to find a method to transform dates into a food form that is closer to the Dietary Guidelines by upcycling the dates into a food product with enhanced nutritional characteristics such as increased protein, e.g., using the dates as a carbon source in the creation of a new food. It would also be desirable if the food product had enhanced organoleptic characteristics (flavor, aroma, taste) compared to the substrates from which it was derived.

In the art, date syrup and date fruit-soaked water has been tried as an alternative carbon source for biomass production of bacteria and fungi. However, the art shows that date extracts suppress the growth of many fungi and bacteria. For example, the growth of the fungus Gilmaniella and the spore forming bacteria Bacillus were suppressed or inhibited by date syrup. See Nazari, (2011) S. African Journal of Biotechnology Vol. 10(3), pp. 424-432. Other studies have shown the ability of date syrup to be used as antimicrobial agent which inhibited many species of bacteria, fungus, and yeasts and the inhibitory properties were observed within a range of concentration from 20 to 50 mg/mL. The art shows that date syrup at 50 mg/mL inhibited bacteria including E. coli, S. aureus, B. subtilis, and fungus including P. digitatum, A. niger and yeasts including C. albicans. There was a significant variation in the antifungal activities date syrup. A. niger was found to be more sensitive than P. digitatum and B. subtilis was found to be more sensitive than E. coli and S. aureus. Antimicrobial activity and preservative properties of date syrup are believed to be associated with phytochemical components such as phenolics and tannins. See Abd-Elhakeim, S. (2018) Middle East J. of Applied Sciences 8(2) 360-369.

Within the vegetarian food sector there are relatively few meat substitutes which aim to provide the proteins which are recommended for daily intake as well as having an appealing texture. Examples of known meat substitutes are tofu, tempeh and mycoproteins known under the name Quorn. The mycoprotein which is used in Quorn is refined from Fusarium venenatum, a mold fungus. Additional meat substitutes based on mold fungi are also under development, such as FY protein (Nature's Fynd).

Fungi are generally considered to include molds, mushrooms, and yeast. Mold refers to a large group of fungi, but do not form a specific taxonomic or phylogenetic grouping but can be found in the divisions Zygomycota and Ascomycota. Mushrooms are another group of fungi, which are mainly Basidiomycetes and partially Ascomycetes, both of which share a same feature—having a macroscopic “fruiting-body, a mushroom”. Fruiting-body is the reproductive organ of the fungus, from which sexual spores are produced and then dispersed either by air or by insects or other animals. Like mushrooms, molds grow as multicellular filaments called hyphae. However, unlike mushrooms, they do not produce a macroscopic fruiting body, i.e., a mushroom.

Although some molds have been developed as human foods, for the most part, with these rare exceptions, molds are not consumed by humans. On the other hand, many types of mushrooms are culinary delicacies and highly prized foods with a long history of consumption.

Thus there remains a need for a way to improve dates and/or date syrup to be a more complete human foods, (upcycle dates) and additionally, find low-cost ways to increase nutrient intake (such as protein) using low-cost protein sources. Also, to-date, there is a need in the art for new meat substitutes with a good flavor (taste, aroma) and texture, particularly from mushroom mycelia. However, it has proven difficult to achieve such products.

### SUMMARY OF THE INVENTION

In one embodiment, the present invention provides methods to produce a composition comprising an edible filamentous fungal biomass, which includes the steps of providing an aqueous media comprising a carbon source comprising an extract of dates; and comprising a nitrogen source; inoculating the media with a filamentous fungal culture, wherein the fungal culture comprises Pleurotus spp., and culturing the filamentous fungal culture in a submerged fungal culture to produce the edible filamentous fungal biomass. In embodiments, the edible filamentous fungal biomass is grown to at least about 25 g/L (dry weight) with a productivity of at least 2.0 g/L/day (dry weight) during the culturing step.

In embodiments, the carbon source comprises an extract of dates or a date syrup, and wherein when the source is date syrup, the date syrup is initially present in the media at a concentration of between about 25 and 35 g/L, or between about 17° Brix and 24° Brix, or between a concentration of between 50 g/L and 110 g/L, between about 40° Brix and 88° Brix.

In embodiments, the nitrogen source in the media may include urea, pea protein, yeast extract, or a combination thereof. In embodiments, the media comprises between about 10 g/L pea protein and about 2 g/L urea. In embodiments, the aqueous media comprises date syrup of between about 25 and 35 g/L, or between about 17° Brix and 24° Brix, pea protein between about 5 g/L and 15 g/L, urea between about 1 g/L and 10 g/L, potassium phosphate between about 0.2 g/L and about 5 g/L and magnesium sulfate between about 0.1 g/L and 2 g/L, and thiamine between about 10 mg/L and 50 mg/L. In embodiments, the culturing step comprises between about seven days and about twelve days. In embodiments, the culturing step comprises a fed-batch culturing step. In embodiments, when the culturing step takes place in a bioreactor, the impeller tip speed is between about 2 and 3 m/s.

In embodiments, the filamentous fungus culture is selected from the group consisting of Pleurotus ostreatus, Pleurotus salmoneostramineus (Pleurotus djamor), Pleurotus eryngii, Pleurotus citrinopileatus, and combinations thereof. In embodiments, the filamentous fungus culture comprises, consists essentially of, or consists of Pleurotus eryngii or Pleurotus ostreatus.

In embodiments, the methods of the invention further include the step of inactivating the edible filamentous fungal biomass by heat treatment. In embodiments, the methods of the invention further comprise the step of harvesting the edible filamentous fungus by dewatering.

In embodiments, the food compositions using the edible filamentous fungal biomass of the invention includes spreads, pastes, prewhipped toppings, custards, coatings, nut butters, frostings, cream filings, confectionery fillings, dairy alternative products such as nondairy milk and nondairy cheeses or spreads, beverages and beverage bases, extruded and extruded/puffed products, meat imitations and extenders, baked goods and baking mixes, granola products, bar products, smoothies and juices, and soups and soup bases.

The invention further includes an edible filamentous fungus composition by the methods disclosed herein, as well as a composition comprising an edible filamentous fungus, wherein the filamentous fungus is Pleurotus spp., which was cultured in a media comprising at least 20 g/L date extract (or at least 16° Brix), and wherein the edible filamentous fungus was produced at a productivity of at least 20 g/L (dry weight).

### BRIEF DESCRIPTION OF THE DRAWINGS

FIG. 1 shows a table with results from a number of pilot scale fermentations to produce Pleurotus ostreatus or Pleurotus eryngii biomass.

### DETAILED DESCRIPTION OF THE INVENTION

The present inventors have unexpectedly found that some select species of filamentous culinary mushroom fungi, including certain culinary species of the mushroom fungus genus Pleurotus, have the ability to grow to high concentrations (with high productivity) using extract of dates such as date syrup. Such results are surprising in view of the art-known anti-microbial activity of date extracts at concentrations used in nutrient media. The present invention provides for high concentrations of date extract in the media (20-25 g/L, e.g., approximately 17° Brix, or above) which have previously shown to be inhibitory to microbes, including many species of bacteria and fungi. However, the present invention provides, in one embodiment, for a particular fungal genus Pleurotus which has the unexpected ability to not only grow at normally inhibitory concentrations of date extract, but to grow to high biomass yield in date extract media.

The present inventors have determined conditions using flask, benchtop and bioreactor scale to maximize productivity while minimizing waste and cost, using date extract materials as, for example, a carbon source.

Accordingly, in an embodiment, the present invention is directed to a method to produce a composition comprising an edible filamentous fungus biomass to maximize productivity (measured as, in one embodiment, as g/L/day) while minimizing waste and cost; which includes the following process parameters, in any order: providing an aqueous media comprising a carbon source comprising an extract of dates, and a nitrogen source; inoculating the media with a filamentous fungal culture, wherein the fungal culture includes Pleurotus spp.; culturing the filamentous fungus to produce the composition comprising filamentous fungus biomass. In an embodiment, the culture is a submerged culture. In an embodiment, the composition comprising the filamentous fungal biomass is grown to at least 25 g dry weight per liter and/or with a productivity of at least 2.5 g biomass solids/L/day (dry weight) during the culturing step. In one embodiment, the composition comprising the filamentous fungal biomass has improved taste, flavor, aroma, and/or color, relative to the starting materials. In one embodiment, the proximate analysis shows at least 20% or at least 40% protein by dry weight.

The filamentous fungal biomass of the invention includes a fungal biomass growing in one of several morphologies, including that of spherical pellets or mycelial clumps. Useful products produced using the composition comprising the filamentous fungi and methods disclosed include, but are not limited to, use in food products, fish feed products, animal feed products, as discussed in more detail hereinbelow. Proteins need not be purified from the filamentous fungal biomass to find utility and usefulness as products and the composition comprising the filamentous fungi can be used directly as a protein source. In certain embodiments, composition comprising the filamentous fungi mycelium described herein comprises significant concentrations of nutrients. In certain embodiments, crude protein accounts for up to 15% to 40% of the untreated desiccated biomass. The mycelium is naturally high in fiber.

Filamentous fungal mycelium biomass by its nature, maintains a texture similar to ground meat with minimal manipulation, and can provide these textural properties to compositions in which they are present. Filamentous fungi mycelium described herein comprises groups of connected cells fused end to end in filaments called hyphae. These hyphae can range from 2-16 microns in diameter and can be centimeters long and can be one single cell thick. These morphologies give the hyphae naturally occurring texture properties similar to muscle fiber as a result of the bundling of the hyphae and the substantial moisture retention capacity of the mycelium. This makes mycelium a perfect candidate for food ingredients and food products.

In certain embodiments, compositions comprising edible filamentous fungus biomass may be processed into a variety of food products including but not limited to meat extenders, meat analogues, cultured meat cell scaffoldings, and other food products requiring textured proteins.

In an embodiment, the inventors have achieved a vegetarian, vegan source of protein, that in one embodiment, transforms low-value e.g., low nutritional value material such as date waste, date extract either normally used for consumption, or normally not used for human consumption due to poor PDCAAS, or due to flavor and taste (sensory) defects, into higher value materials, e.g., compositions comprising edible filamentous fungus biomass, which are more highly prized for consumption and/or have better nutritional values. Thus compositions comprising filamentous fungal biomass achieved by culturing and/or myceliation according to the present invention, may have an improved value based on improved organoleptics, change in color, improved nutritionals (including protein composition and/or percentage) relative to the starting materials or the media components.

In particular embodiments, provided herein are methods for producing good quality protein food ingredients. In certain embodiments, the methods comprise the steps of culturing filamentous fungus biomass in growth medium; harvesting filamentous fungal biomass; optionally, and processing the harvested filamentous fungal biomass. In certain embodiments, the methods comprise the steps of culturing filamentous fungi in a growth medium; optionally supplementing the growth medium to form a mixed fungal biomass slurry; harvesting filamentous fungal biomass; optionally, and processing the harvested filamentous fungal biomass to form the food ingredient.

In the culturing step, the filamentous fungus can be cultured according to standard techniques. The culturing typically comprises growing the filamentous fungus in a growth medium. In certain embodiments, the culture is batch culture, fed-batch culture, or continuous culture. The growth medium includes the ingredients described below. Additional additives can be provided according to the judgment of the practitioner in the art. Culture conditions are within the skill of those in the art including culture volume, temperature, agitation, oxygen levels, nitrogen levels, carbon dioxide levels, and any other condition apparent to those of skill.

In certain embodiments, pure oxygen is used in the aeration of the fermentation.

The fungal fermentation can operate with a wide pH range. In certain embodiments the pH is between about pH 4 and about pH 8.5, between about pH 4.5 and about pH 7.5, between about pH 5 and about pH 7, between about pH 5.5 and about pH 6.5, or between about pH 5.8 and about pH 6.3.

The compositions of the invention may also include one or more additional components. In certain embodiments materials such as high protein materials comprising e.g., plant protein, yeast protein, amino acids, and the like are added to the composition comprising filamentous fungus biomass at any time during the manufacturing process. In one embodiment, plant protein (e.g., pea protein compositions) are present in the growth media for the filamentous fungus and are incompletely consumed by the fungus and are present in the produced composition comprising filamentous fungus biomass in amounts of between 5% and 95%, or in amounts of between 10% and 80%, or in amounts of between 20% and 70%, or in an amounts of between 30% and 60%; or in amounts of between 10% and 30%, of the composition. Alternatively, these materials can be added to the harvested fungal biomass, either before any dehydration steps, e.g., added to a slurry of the fungal biomass having a water content of 60-85%, the fungal biomass of a water content of 60-75%, the fungal biomass of a water content of 50-75%, the fungal biomass of a water content of 50-65%, or fungal biomass with other water content. These materials may be blended with the fungal biomass described herein and then further de-watered, de-hydrated, or processed into the dried textured ingredients described herein. Alternatively, the materials may be added to the compositions comprising filamentous fungus biomass following any dehydration/drying steps.

### Growth Medium

The aqueous media may include a carbon source that may comprise, consist of, or consist essentially of an extract of dates. The term “dates” include fruit of the date palm Phoenix dactylifera. An “extract of dates” can include dates and any product produced from date using aqueous extraction techniques, such as date juice concentrates (spread, syrup and liquid sugar), date pastes, and the like. In an example of one embodiment to produce pastes, dates are steamed, destoned, macerated, and converted to a semi-solid form known as paste with approximately 20-23% moisture content and a water activity below 0.6. In an example of one embodiment to produce syrup, the fruits are mixed with water and heated for around 1 h at 50° C. and the main component, sugars, are then extracted. Dates are rich in sugar ranging from 65% to 80% on dry weight basis mostly of inverted form (glucose and fructose). Fresh varieties have a higher content of inverted sugars, the semi dried varieties contain equal amounts of inverted sugars and sucrose, while dried varieties contain higher sucrose. Water content is between 7% (dried) and 79% (fresh) depending on variety.

Date waste from any of the processes described herein to produce date extract can be utilized to produce mycelia by using the waste as solid-state media; or alternatively, by using date waste as a source of fiber for further processing the filamentous fungal biomass into different food forms, such as texturized protein, as described hereinbelow.

In one embodiment of the present invention, the date extract comprises, consists of, or consists essentially of a date syrup. Date syrup has the following standard specifications: minimum Brix of 70°, pH value range of 4.2-6, maximum ash content of 2%, and a minimum reducing sugars of 58% (INSO 5075, 2013). The media that contains the date extract as described above can have date extract present in amounts as defined by the following measurements. One measurement may be by grams of date extract or date syrup, per liter of media. However, different date extracts will contain different amounts of monosaccharides. Date extracts can be standardized by ° Brix measurement. Degrees Brix (symbol ° Bx) measures the sugar (monosaccharides) content of an aqueous solution. One degree Brix is equivalent to 1 gram of sugar in 100 grams of solution. Generally, for fruit juices, 1.0 degree Brix is denoted as 1.0% sugar by mass.

In one embodiment, date extract is used according to its Brix value. For example, if a date extract is a date syrup having 80° Brix, then it has 80 g of sugar (monosaccharides) per 100 g (or 100 mL) of water. Using 30 g of an 80° Brix solution in 1 L of media results in an amount of sugar (monosaccharides) of about 24 g/L or about 24° Brix. Accordingly, in the present invention, using date extracts of different ° Bx values can be “standardized” to a final concentration in the aqueous media of g/L sugar (monosaccharides) by the starting ° Bx value of the date extract.

Accordingly, the present invention may include an amount of date extract that is standardized to between about 5 g/L sugar (monosaccharides) to about 50 g/L sugar (monosaccharides). In embodiments, the amount of date extract to add is between about 10 g/L sugar (monosaccharides) to about 40 g/L sugar (monosaccharides), between about 15 g/L sugar (monosaccharides) and about 35 g/L sugar (monosaccharides), between about 20 g/L sugar (monosaccharides) and about 30 g/L, or between about 22 g/L sugar (monosaccharides) and about 28 g/L sugar (monosaccharides), or between about 24 g/L sugar (monosaccharides) and about 26 g/L sugar (monosaccharides). For a date syrup of about 85° Brix, the amount to use can be between about 10 g/L and about 50 g/L, between about 15 g/L and about 45 g/L, between about 20 g/L and about 40 g/L, between about 25 g/L and about 35 g/L, between about 27 g/L and about 33 g/L, between about 29 g/L and about 31 g/L.

Optionally, the media also comprises a nitrogen source. In one embodiment, the nitrogen source is a protein such as a protein concentrate or isolate from a vegetarian source, plant source, a mycoprotein, a yeast extract and the like. Appropriate proteins include yeast extract, pea protein, rice protein, soybean, oat protein, hemp protein, chickpea flour, chia powder, cyanobacteria or algal protein, and the like. In one embodiment, the protein is a, or is derived from, a pulse (seed) from a legume, such as pea, chickpea, lentils, lupins, common beans (kidney, pinto). In embodiments, the protein(s) are produced from pea, rice, chickpea or a combination thereof. In one embodiment, the media may comprise pea protein, chickpea, and corn gluten meal. Other, lower quality protein isolates and concentrates (or whole unprocessed, optionally milled) may also be used such as fava bean, red beans, broad beans, sunflower meal, canola meal, DDGS meal, copra meal, lupin meal, lemna meal, or corn gluten meal. In one embodiment, the protein is a low-quality protein. A “low-quality protein,” includes vegetable proteins which typically have lower PDCAAS scores than meats, and can include proteins with PDCAAS scores below 0.60, for example, indicating a deficiency of one or more essential amino acids, typically low in lysine (corn) or low in tryptophan (beans). In embodiments, a “low-quality protein” also includes proteins, that, in embodiments, refer to plant proteins that is typically not suitable for human ingestion due to such factors as organoleptic challenges including undesirable flavors, aromas and/or tastes, which may be improved by a biomass of the present invention.

The protein material to add to the media, itself can be unprocessed (or, optionally milled) or a concentrate or isolate of at least about 2 g (dry weight) per L, at least about 4 g/L, at least about 6 g/L, at least about 8 g/L, at least about 10 g/L, at least about 12 g/L, at least about 14 g/L, at least about 16 g/L, at least about 18 g/L, at least about 20 g/L, at least about 22 g/L. In one embodiment, the amount to use is between about 10 g/L and about 20 g/L, between about 12 g/L and about 18 g/L, between about 14 g/L and about 16 g/L.

In other embodiments, the nitrogen source can comprise an organic or inorganic nitrogen source, such as, for example, ammonium chloride, ammonium phosphate dibasic, ammonium sulfate, urea, and/or combinations thereof, and can be used in equivalent amounts (on a nitrogen basis) as the proteins provided above.

In certain embodiments, the nitrogen source can comprise mixtures of pea protein and urea. In embodiments, the mixture can comprise from about 1 g/L of pea protein to about 20 g/L or more of pea protein, or from about 5 g/L to about 15 g/L of pea protein, or from about 8 g/L to about 12 g/L of pea protein, or about 10 g/L pea protein. In embodiments, pea protein in the media can comprise between about 1 g/L and 6 g/L, between about 1 g/L and 4 g/L or between about 1 g/L and 3 g/L, or about 2 g/L. In embodiments, the mixture can comprise between about 0.5 g/L urea to about 10 g/L urea, or between about 1 g/L to about 6 g/L urea, or between about 1.5 g/L urea to about 3 g/L urea. In embodiments, the mixture comprises, consists of, or consists essentially of about 10 g/L pea protein and about 2 g/L urea.

In some embodiments, the growth medium comprises one or more plant substrates selected from pea fiber, other plant fibers, gum arabic, natural flavors, texturized pea protein, texturized wheat protein, texturized soy protein, soy protein, wheat starch, wheat protein, pea protein, spices, safflower oil, sunflower oil, olive oil, other oils, oat bran, oat flour, legumes, beans, lentils, lentil powder, bean powder, pea powder, yeast extract, nutritional yeast (immobilized dried yeast), molasses, honey, cane sugar, mushroom powder, white button mushroom powder, shiitake mushroom powder, chickpeas, bamboo fiber, cellulose, isolated oat product, isolated pea product, pea protein, rice protein, fermented rice extract, corn starch, potato starch, kombu extract, algae, potato protein, albumin, pectin, silicone dioxide, food starch, mixed tocopherols (vitamin E), coconut oil, sunflower oil, safflower oil, rapeseed oil, canola oil, dextrose, vegetable glycerin, dried yeast, citrus extract, citrus fiber, beet pulp, beet juice, beet juice extract, turmeric, mushroom extract, shiitake mushroom stems, shiitake mushrooms, white button mushrooms, tofu, soy fiber, soy hydrolysate, yeast extract, seaweed, malted barley, malt extract, yeast extract, whole cell yeast, lentils, black beans, pinto beans, beans, legumes, and any combination thereof. In preferred embodiments, the plant biomass is potato or corn stillage.

In certain embodiments, the growth medium further comprises one or more carbohydrates. In certain embodiments the one or more carbohydrates are selected from glucose, sucrose, dextrose, starch, maltose, and any combination thereof.

In embodiments, a pea protein concentrate is useful for the present invention. A protein concentrate is made by removing the oil and retaining the meal or may be a whole ingredient. Typically, protein concentrations in such products are between 25-99%. The process for production of a protein isolate typically removes most of the non-protein material such as fiber and may contain up to about 90-99% protein.

In one embodiment, mixtures of any of the one or more proteins disclosed can be used to provide, for example, favorable qualities, such as a more complete (in terms of amino acid composition) low-quality protein composition. In other embodiments, low-quality protein compositions may be supplemented by using amino acids in purified or partially purified form.

In embodiments, the carbon to nitrogen ratio can vary between 7 and 14 molar ratio of carbon to molar ratio of nitrogen. In one embodiment, the ratio is between about 9 and 11 molar carbon to nitrogen.

In some embodiments, the media may be partially dissolved, and/or partially suspended, and/or partially colloidal. However, even in the absence of complete dissolution of, positive changes may be affected during culturing. In one embodiment, the ingredients in the aqueous media are kept as homogenous as possible during culturing, such as by ensuring agitation and/or shaking.

In certain embodiments, the growth medium is supplemented with one or more additive components. The additive components might facilitate growth of the filamentous fungi, they might add nutrients to the resulting food product, or they might do both. In certain embodiments, the additive components comprise one or more carbohydrates (simple and/or complex), nitrogen, vitamins such as thiamine, minerals, fats, proteins, or a combination thereof.

In certain embodiments, the additive components comprise one or more oils. In certain embodiments, the one or more oils are selected from the group consisting of grapeseed oil, safflower oil, sunflower oil, olive oil, coconut oil, flaxseed oil, avocado oil, soybean oil, palm oil, canola oil, and combinations thereof.

In certain embodiments, the one or more additives comprise one or more salts. In certain embodiments, the one or more salts consist of elements selected from the group consisting of C, Zn, Co, Mg, K, Fe, Cu, Na, Mo, S, N, P, Ca, Cl, and combinations thereof.

In certain embodiments the salts comprise one or more of the following salts: ammonium nitrate, mono-potassium phosphate, di-potassium phosphate, di-ammonium phosphate, ammonium phosphate, potassium nitrate, magnesium sulfate heptahydrate, calcium chloride dehydrate, zinc sulfate heptahydrate, iron sulfate hexahydrate, copper sulfate pentahydrate, manganese sulfate, and combinations thereof.

In embodiments, the aqueous media further comprises, consists of, or consists essentially of at least one exogenously-added additional amino acid, purified or partially purified. The amino acid may be used to supplement the low-quality protein material where its amino acids are low, e.g., to create a material with a better PDCAAS score by adding, e.g., lysine, sulfur amino acids, and/or tryptophan. In an embodiment, the at least one amino acid may be at least one branched chain amino acid (“BCAA”) which is exogenously added to the high-protein material to increase the BCAA content. Examples of sources include Ajinomoto AMINO L40, which contains 9 essential amino acids (L-leucine, L-lysine, L-valine, L-isoleucine, L-threonine, L-phenylalanine, L-methionine, L-histidine, L-tryptophan).

Excipients may also include peptones/proteins/peptides, as is known in the art to support fungal growth. These are usually added as a mixture of protein hydrolysate (peptone) and meat infusion. Many media have, for example, between 1% and 5% peptone content, and between 0.1 and 5% yeast extract and the like.

In one embodiment, excipients include for example, yeast extract, malt extract, maltodextrin, peptones, and salts such as diammonium phosphate and magnesium sulfate, as well as other defined and undefined components such as potato or carrot powder. In some embodiments, organic forms of these components may be used. Excipients may also optionally comprise, consist of, or consist essentially of citric acid and an anti-foam component.

The method may also comprise the optional step of sterilizing the aqueous media prior to inoculation by methods known in the art, including steam sterilization and all other known methods to allow for sterile procedure to be followed throughout the inoculation and culturing steps to enable culturing and myceliation by pure fungal strains. Alternatively, the components of the media may be separately sterilized and the media may be produced according to sterile procedure. In embodiments, to reduce the production of Maillard reactions which can result in darkening of the produced compositions comprising an edible filamentous fungal biomass, carbon-sources can be sterilized or autoclaved separately from the nitrogen sources, in whole or in part.

The filamentous fungal cultures, prior to the inoculation step, may be propagated and maintained as is known in the art. In some embodiments, liquid cultures used to maintain and propagate fungi for use for inoculating the one or more ingredients as disclosed in the present invention include undefined agricultural media with optional supplements as a motif to produce culture for the purposes of inoculating solid-state material or larger volumes of liquid. In some embodiments, liquid media preparations are made as disclosed herein. Liquid media can be also sterilized and cooled similarly to agar media. Bioreactors provide the ability to monitor and control aeration, foam, temperature, and pH and other parameters of the culture and as such enables shorter myceliation times and the opportunity to make more concentrated media.

In one embodiment, the filamentous fungi for use for inoculating the one or more ingredients disclosed in the present invention may be produced as a submerged liquid culture and agitated on a shaker table, or may be produced in a shaker flask, by methods known in the art and according to media recipes disclosed in the present invention. In one embodiment, the fungal component may be produced from a glycerol stock, by a simple propagation motif of Petri plate culture to 0.5 to 4 L Erlenmeyer shake flask to 50% glycerol stock. Petri plates can comprise agar in 10 to 35 g/L in addition to various media components. Conducted in sterile operation, chosen Petri plates can be propagated into 0.5 to 4 L Erlenmeyer flasks (or 250 to 1,000 mL Wheaton jars, or any suitable glassware) for incubation on a shaker table or stationary incubation. In one embodiment, the shaking is anywhere from 40-160 RPM depending on container size and, with about a 1″ swing radius.

Inoculum for bioreactor fermentations may be prepared by methods known in the art for a particular species. In one embodiment, for Pleurotus eryngii inoculum, the seed flasks (initially inoculated with 25 g/L date syrup or higher amounts) may be used for inoculating once glucose concentrations reach 3-4 g/L or lower, which may require 7 to 30 days of culture. The pH of the inoculum upon use, at any sugar concentration is typically between 5.5-6.5. Once these parameters are reached, the inoculum is typically briefly blended with a WARING-type blender to break up mycelia pellets and provide a homogenous slurry. The refractive index of the blended inoculum is optionally from 0.2-1.0 OD when diluted to linear range and have a plate count of anywhere from 200,000-1,200,000 cfu/mL.

The culturing step of the present invention may be performed by methods (such as sterile procedure) known in the art and disclosed herein and may be carried out in a fermenter, shake flask, bioreactor, or other methods. In an embodiment the incubation temperature is 70-90° F. Liquid-state fermentation agitation and swirling techniques as known in the art are also employed which include mechanical shearing using magnetic stir bars, stainless steel impellers, injection of sterile high-pressure air, the use of shaker tables and other methods such as lighting regimen, batch feeding or chemostatic culturing, as known in the art.

The major factors for scale-up for bioreactors and microorganism growth are the agitation rate, linear velocity, OUR (oxygen uptake rate), kLa (oxygen transfer rate), configuration (size and diameter) of the fermenter, and the diameter and shape of the agitation blade. However, the conditions for optimally growing the filamentous fungal culture of the present invention are not well understood. For example, process parameters for filamentous fungal growth using submerged fermentation usually are optimized for compact and small pellets due to rheological issues. On the other hand, optimal productivity can be associated with less dense and larger morphology. The morphological state of filamentous fungi has a large impact on process performance in a bioreactor. For example, in free mycelia, high biomass concentrations result in highly viscous fermentation media, resulting in issues with gas-liquid mass transfer, liquid mixing and a generally complex rheology. It is known in the art that enhanced productivity of mycelia and clumps requires access to oxygen and substrates. The art teaches to optimize bioreactor conditions to result in pellet which is a large agglomeration of productive sections that have open access to substrates and oxygen, although this structure is associated with highly viscous fermentation media, resulting in issues with gas-liquid mass transfer, liquid mixing and complex rheology. On the other hand, less viscous media results from smaller and more compact pellets. Therefore, morphology is optimized to balance rheological requirements for the bioprocess on the one hand and ideal pellet compactness for enhanced productivity.

In embodiments of the present invention, as agitation is known to generally result in smaller pellets but is taught by the art to result in risk of shear stress to the organism (resulting in slower growth rates) and even rupture of the cells of the organism, in addition, higher agitation can result in increased foaming, thus the art teaches an aeration rate of 0.5 VVM or below, with an agitation rate of 150-200 rpm of the impeller to minimize these issues. See, e.g., J. H. Seo et al, Korean J. Chem. Eng., 24(5), 800-805 (2007). In the present invention, it was unexpectedly found that relatively high agitation rates make a difference in the growth rates of the organism for the present invention.

Agitation rates may be scaled up from benchtop to pilot to production scales in several different methods, each with different advantages and disadvantages. One method involves calculating the tip speed of the edge of the impeller blade and keeping it constant at scale. The impeller tip speed is the product of the impeller diameter, x, and shaft rpm. Another method is setting the power per volume constant for each scale. The agitator motor power is measured and divided by the volume of the bioreactor. The power requirement of a larger vessel can be estimated by using this ratio. A more advanced method of agitation scale-up involves the use of dimensionless numbers which allow for comparisons of power drawn from the impeller and level of mixing. The power number (Np) is a function of electrical power (P), fluid density (p), shaft rotations per second (N), and impeller diameter (D) and is defined as

$${N}_{p} = \frac{P}{ρ {N}^{3} {D}^{5}} .$$

The mixing powerless number (Nq) is a function of the impeller pumping rate (Q) and is defined as

$${N}_{q} = \frac{Q}{{ND}^{3}} .$$

Aeration is closely linked with agitation and, in some cases, too high of an air flow rate can flood the impeller and cause poor mixing. One method of aeration scale up is to keep the volumetric gas flow rate constant at scale. The volumetric gas flow rate is defined as the quotient of the air flow rate and the fermenter volume. This parameter is often expressed as VVM's or vessel volumes per minute. Another method includes using pilot data to fit a mass-transfer correlation using the following equation:

$${K}_{l} a = {K}^{′} {\frac{P}{V}}^{a} {{U}_{s}}^{b}$$

where Kₗα is the mass-transfer coefficient, P is the agitator power, V is the vessel volume, Uₛ is the superficial gas velocity, and a, b, and K′ are constants for the model to fit. An example of bioreactor scale-up from data from a benchtop bioreactor is detailed below in Table 1:

TABLE 1

| | | |
|---|---|---|
| Parameter | Benchtop | Pilot |

| | | | | |
|---|---|---|---|---|
| Working Volume | 4 | L | 40 | L |

| | | |
|---|---|---|
| Agitator Type | Rushton | Rushton |
| Number of Impellers | 2 | 2 |

| Impeller Diameter | 0.13 | m | 0.20 | m |
|---|---|---|---|---|
| Impeller Circumference | 0.40 | m | 0.64 | m |
| Shaft RPM | 120 | | 75 | |
| Impeller tip speed | 0.80 | m/s | 0.80 | m/s |
| Air Rate | 3 | Nlpm | 30 | Nlpm |

| | | |
|---|---|---|
| VVM | 0.75 | 0.75 |

In embodiments, the aeration rate is at least 0.5 vvm, at least 0.6 vvm, at least 07 vvm, at least 1 VVM, at least 1.5 VVM, at least 2 VVM, at least 3 VVM, at least 4 VVM, at least 59 VVM, at least 6 VVM, at least 8 VVM, at least 10 VVM, at least 15 VVM, at least 20 VVM or more. The scale of the reactor will inform an appropriate VVM as the residence time of air bubbles in the reactor will differ depending on the size of the reactor; for example, for a small reactor, high VVM are appropriate (10 or 20 VVM or more) due to shorter residence time of air bubbles, whereas VVM in a larger reactor will be significantly lower while having the same aeration effect.

In embodiments, it was surprisingly found that higher than taught agitation rates in a bioreactor for the present invention's culturing step, makes a positive difference in the growth rates of the organism for the present invention. For example, the agitation rate of 120 rpm (shaft rotation), using the dimensions of the bioreactor as shown in Table 1, translates to an impeller tip speed of 0.8 meters/second (m/s) according to standard calculations. It was found that increasing the agitation rate to 400 rpm (impeller tip speed of 2.66 m/s) in a 7 L bioreactor (4 L working volume) increased the productivity as shown below in the Examples. Accordingly, in some embodiments, the present invention employs in a bioreactor, a impeller tip speed of between about 0.5 to about 10 m/s, or a impeller tip speed of between about 1 to about 6 m/s, or a impeller tip speed of between about 2 to 3 m/s. Alternatively, the impeller tip speed is at least about 1 m/s, at least about 2 m/s, at least about 2.5 m/s, at least about 3 m/s, at least about 4 m/s, or at least about 5 m/s. However, as noted, too high an impeller tip speed (greater than about 10 m/s or so) can result in shear stress to the organism resulting in lower productivity.

Bioreactor cultivation may be carried out in a batch or continuous manner.

In the culturing step, the filamentous fungus can be cultured according to standard techniques. The culturing typically comprises growing the filamentous fungus in a growth medium as described herein. Culture conditions are within the skill of those in the art including culture volume, temperature, agitation, oxygen levels, nitrogen levels, carbon dioxide levels, and any other condition apparent to those of skill.

The fungal fermentation can operate with a wide pH range. In certain embodiments the pH is between about pH 4 and about pH 8.5, between about pH 4.5 and about pH 7.5, between about pH 5 and about pH 7, between about pH 5.5 and about pH 6.5, or between about pH 5 and about pH 6.

In one embodiment, culturing step is carried out in a bioreactor which is ideally constructed with a torispherical dome, cylindrical body, and spherical cap base, jacketed about the body, equipped with a magnetic drive mixer, and ports to provide access for equipment comprising DO, pH, temperature, level and conductivity meters as is known in the art. Any vessel capable of executing the methods of the present invention may be used. In another embodiment the set-up provides 0.1-5.0 ACH. Other engineering schemes known to those skilled in the art may also be used.

The reactor can be outfitted to be filled with water. The water supply system is ideally water for injection (WFI) system, with a sterilizable line between the still and the reactor, though RO or any potable water source may be used so long as the water is sterile. In one embodiment the entire media is sterilized in situ while in another embodiment concentrated media is sterilized and diluted into a vessel filled water that was filter and/or heat sterilized. In another embodiment, high temperature high pressure sterilizations are fast enough to be not detrimental to the media. In one embodiment the entire media is sterilized in continuous mode by applying high temperature between 130° and 150° C. for a residence time of 1 to 15 minutes. Once produced with a working volume of sterile media, the tank can be mildly agitated and inoculated. Either as a concentrate or whole media volume in situ, the media can be heat sterilized by steaming either the jacket, chamber or both while the media is optionally agitated. The medium may optionally be pasteurized instead.

In one embodiment, the reactor is used at a large volume, such as in 500,000-7,000 L working volume bioreactors. When preparing material at such volumes the culture must pass through a successive series of larger bioreactors, any bioreactor being inoculated at 0.5-15% of the working volume according to the parameters of the seed train. A typical process would pass a culture from master-culture, to Petri plates, to flasks, to seed bioreactors to the final main bioreactor when scaling the method of the present invention. To reach large volumes, 3-4 seeds may be used. The media of the seed can be the same or different as the media in the main. In one embodiment, the fungal culture for the seed is a one or more ingredients as defined herein, to assist the fungal culture in adapting to one or more ingredients media in preparation for the main fermentation. In one embodiment, foaming is minimized by use of antifoam on the order of 0.5 to 2.5 g/L of media, such as those known in the art, including insoluble oils, polydimethylsiloxanes and other silicones, certain alcohols, stearates and glycols. In one embodiment, lowering pH assists in culture growth, for example, pH may be adjusted by use of citric acid or by any other compound known in the art, but care must be taken to avoid a sour taste for the myceliated one or more ingredients. The pH may be adjusted to between about 4.5 and 5.5, for example, to assist in growth. In some embodiments, the pH is higher, between about 5.5 and 6.5, or, between 5.8 and 6.3, either as initial conditions or throughout the fermentation. In some embodiments, the pH remains between 5.8 and 6.3 either without adjustment or with adjustments.

Another method for producing the edible filamentous fungal biomass include use of fermentation technology known as “airlift” bioreactors. Bioreactors with a relatively low mixing intensity (airlift bioreactor) can be more effective for this process than devices with mechanical mixing. An example of a laboratory-scale batch system is a 4-L airlift bioreactor (Belach Bioteknik, Stockholm, Sweden) having a nominal working volume of 3.5 L. The riser and downward pattern can be applied for the reactor flow and circulation. Sample conditions for fermentation include inlet air passed through a 0.2-μm pore sterile polytetrafluoroethylene filter and entered the bottom of the reactor filled with media and then inoculated. Antifoam (after sterilization) and fungal inoculum may be added at the inlet top of the running reactor at the beginning of cultivation and the antifoam gradually added to control foam throughout the cultivation. The airflow, temperature, and pH for example can be 0.75 VVM (volume of air per volume of culture per minute), 26° C., and 5.5±0.02 (adjusted by sodium hydroxide (NaOH) or phosphoric acid (H₃PO₄)), respectively. All media can be sterilized in an autoclave at 121° C. for 30 min.

The compositions comprising fungal biomass can be harvested by pouring out the cultivation medium through a fine mesh (1 mm² pore area) stainless steel sieve after cultivation and washed with tap water until a clear filtrate was observed, freeze-dried and stored at minus 20° C. for further analysis of crude protein, total fat, amino acids, fatty acids, ash, and cell wall contents. The biomass amount can be gravimetrically measured and reported as grams of dried biomass per liter of the spent liquor.

In one embodiment, a preparation of a filamentous fungal component for use for inoculating an aqueous media was produced and used to create the edible filamentous fungal biomass.

It was found that not all fungi are capable of growing in media as described herein. Fungi useful for the present invention are from the higher order Basidio- and Ascomycetes, e.g., filamentous fungi. Particularly suitable for the present invention include Pleurotus species. Examples of appropriate Pleurotus species to use comprise, consist of, or consist essentially of, for example, species of P. ostreatus clade: P. ostreatus (oyster or pearl oyster mushroom), Pleurotus spodoleucus (Korean oyster), P. florida, P. pulmonarius (phoenix or Indian oyster mushroom), P. columbinus, P. sapidus, P. populinus, P. eryngii (king oyster mushroom), P. ferulae, P. fossulatus, P. nebrodensis, P. abieticola, P. albidus; also the P. djamor-cornucopiae clade, such as P. cornucopiae (branched oyster mushroom), P. citrinopileatus (golden oyster mushroom), P. euosmus (tarragon oyster mushroom). P. djamor (pink oyster mushroom), P. flabellatus, P. salmoneostramineus, P. salmonicolor, P. opuntiae, P. calyptratus; and P. cystidiosus clade including P. cystidiosus (abalone mushroom), P. abalonus, P. fuscosquamulosus, P. smithii, P. dryinus, P. tuber-regium (king tuber mushroom) P. australis (brown oyster mushroom), P. purpureo-olivaceus, P. P rattenburyi. In embodiments Pleurotus comprises, consists of, or consists essentially of Pleurotus ostreatus, Pleurotus salmoneostramineus, Pleurotus djamor, Pleurotus eryngii, or Pleurotus citrinopileatus. In another embodiment, the fungi is M. esculenta. Additional suitable strains for the present invention include Agrocybe aegerita, Volvariella volvacea, Termitomyces albuminosus, and Laetiporus sulphureus. Fungi may be obtained commercially, for example, from The Pennsylvania State University Mushroom Culture Collection, available from the College of Agriculture Sciences, Department of Plant Pathology and Environmental Microbiology, 117 Buckhout Laboratory, The Pennsylvania State University, University Park, Pennsylvania, USA 16802.

The compositions comprising cultured filamentous fungi can be harvested according to any standard technique. The methods include any harvesting technique deemed useful to the practitioner in the art. Useful techniques include centrifugation, pressing, screening, and filtration. In certain embodiments, the compositions comprising filamentous fungi are de-watered and separated by centrifugation. In certain embodiments, the compositions comprising filamentous fungi are de-watered and separated via screw press. In certain embodiments fungi are de-watered and separated via de-watering vibratory screen. In certain embodiments fungi are de-watered via a fluidized bed dryer. In certain embodiments, the compositions comprising filamentous fungi are washed to remove excess growth medium. In certain embodiments, the compositions comprising filamentous fungi are not washed.

Determining when to end the culturing step and to harvest the filamentous fungal biomass composition, which according to the present invention, to result in an edible filamentous fungal biomass, can be determined in accordance with any one of a number of factors as defined herein, such as, for example, visual inspection of mycelia, microscope inspection of mycelia, pH changes, changes in dissolved oxygen content, amount of biomass produced, and/or assessment of taste profile, flavor profile, or aroma profile.

In one embodiment, the harvest is determined by when the culture reaches a stationary phase of growth. In another embodiment, production of a certain amount of biomass may be the criteria used for harvest. For example, biomass may be measured by filtering, such through a filter of 10-1000 μm. In one embodiment, harvest can occur when the dissolved oxygen reaches about 10% to about 90% dissolved oxygen, or less than about 80% of the starting dissolved oxygen. Additionally, mycelial products may be measured as a proxy for mycelial growth, such as, total reducing sugars (usually a 40-95% reduction), β-glucan and/or ergosterol formation. Other indicators include small molecule metabolite production depending on the strain or nitrogen utilization (monitoring through the use of any nitrogenous salts or protein, may continue to culture to enhance the presence of mycelial metabolites).

In one embodiment, harvest conditions are at least partially determined by glucose level, e.g., glucose of 4 g/L or less.

Date syrup contains fructose. As determined by the present inventors, the fructose component of date syrup, while supporting growth of the filamentous fungal biomass, is not as efficient as supporting the growth as the glucose component. However, in some embodiments, it is desirable, for example, efficiency purposes, to promote fructose consumption by the filamentous fungal biomass. Thus, in an embodiment, the solution found by the present inventors is adjust the harvest criteria such that at the completion of fermentation (as determined by the parameters disclosed herein, including low glucose levels), that the fermentation be allowed to continue (without any feeding steps) for an additional time, to allow the filamentous fungal biomass to consume or partially consume the fructose component of the dates. For example, the additional time may be from 8 to 48 hours of fermentation, or from about 10 to 36 hours of fermentation, or about 12 to 30 hours of fermentation time. In an embodiment, the additional time is about 24 hours.

In an embodiment, the filamentous fungal biomass is grown to at least 25 g dry weight per liter and/or with a productivity of at least 2.5 g/L/day (dry weight) during the culturing step. Under the conditions disclosed in the invention, the fungal cultures are capable of rapid, high density cell growth and are capable of achieving a cell density (measured as dry weight per liter of media) of at least about 10 g/L, at least about 15 g/L, at least about 20 g/L, at least about 25 g/L, at least about 30 g/L, at least about 35 g/L, at least about 40 g/L, at least about 45 g/L or at least about 50 g/L. Or, the fungal cultures are capable of achieving a cell density of from about 15 g/L to about 40 g/L, from about 20 g/L to about 35 g/L, from about 25 g/L to about 30 g/L. Alternatively, the filamentous fungal biomass is grown under conditions as disclosed herein, which result in measured productivity (g dry weight biomass yield) of at least 1.5 g/L/day, at least 2 g/L/day, at least 2.5 g/L/day, at least 3 g/L/day, at least 3.5 g/L/day, at least 4 g/L/day, at least 4.5 g/L/day, at least 5 g/L/day, at least 5.5 g/L/day, at least 6 g/L/day, as an overall growth rate during the fermentation.

In one embodiment, the proximate analysis of the filamentous fungal biomass grown by methods of the invention comprise at least 20% protein by dry weight, at least 25% protein by dry weight, at least 30% protein by dry weight, at least 35% protein by dry weight, at least 40% protein by dry weight, at least 45% protein by dry weight, at least 50% protein by dry weight, or at least 55% protein by dry weight, but no more than 70% protein by dry weight in all cases.

In another embodiment, the filamentous fungal biomass exhibits an improved PDCAAS. In an embodiment, it is possible to achieve a PDCAAS score that improves on the input materials. The improvement can be by about 5% increase, 10% increase, 15% increase, or 20% increase, for example.

Culturing time for the main fermentation can be from 1 day to about 30 days. In one embodiment, the culturing time in the main fermentation is between about 5 days and about 20 days, or between about 7 days and 15 days.

Harvest includes obtaining the edible filamentous fungal biomass which is the result of the culturing step. After harvest, cultures can be processed according to a variety of methods. In one embodiment, the filamentous fungal biomass is pasteurized or sterilized. In one embodiment, the filamentous fungal biomass is then dried according to methods as known in the art. Additionally, concentrates and isolates of the material may be produced using variety of solvents or other processing techniques known in the art.

In one embodiment, the harvest is determined by when the culture reaches a stationary phase of growth. The biomass may be separated from the media by filtration (on a filter press or vacuum filter) or separation and spray drying of biomass. In one embodiment the material is pasteurized or sterilized, dried and powdered by methods known in the art. Drying can be done in a desiccator, vacuum dryer, conical dryer, spray dryer, fluid bed, infrared dryer, or any method known in the art. Preferably, methods are chosen that yield a dried filamentous fungal biomass composition (e.g., a powder) with the greatest digestibility and bioavailability. The dried one or more filamentous fungal biomass composition can be optionally blended, pestled, milled or pulverized, or other methods as known in the art.

The cultured composition comprising filamentous fungus biomass can be harvested according to any standard technique. The methods include any harvesting technique deemed useful to the practitioner in the art. Useful techniques include centrifugation, pressing, screening, and filtration. In certain embodiments, the composition comprising filamentous fungus biomass is de-watered and separated by centrifugation. In certain embodiments, the composition comprising filamentous fungus biomass is de-watered and separated via screw press. In certain embodiments the composition comprising filamentous fungus biomass is de-watered and separated via de-watering vibratory screen. In certain embodiments the composition comprising filamentous fungus biomass is de-watered via a fluidized bed dryer. In certain embodiments, the composition comprising filamentous fungus biomass is washed to remove excess growth medium. In certain embodiments, the composition comprising filamentous fungus biomass is not washed.

In certain embodiments, the harvested filamentous fungal biomass is processed for further use. The processing technique can be any processing technique apparent to the practitioner of skill.

In certain embodiments, the composition comprising filamentous fungus biomass is sized according to the requirements of the ingredients described herein. Biomass released from de-watering processes can be cake-like as it is similar to fruit, paper, or other pulp upon de-watering. This cake can be broken apart, shredded, chopped, sliced, diced, sieved, and further reduced to desired size using conventional sizing equipment before or after de-hydration. The biomass can be molded, pressed, rolled, extruded, compacted, or manipulated in other ways known to a person with skill in the art of sizing food materials, either before de-watering, during, de-watering, after de-watering, before de-hydration, during de-hydration, or after de-hydration. In certain embodiments, the biomass is sized to yield shreds of defined or desired size parameters.

In certain embodiments, the composition comprising filamentous fungus biomass comprises highly dispersed filamentous cell structures. Without being bound by theory, upon removing water using gravity, pressure, compaction, vacuum suction, centrifugation, or other methods known to those skilled in the art of de-watering, filamentous cellular strands can interlock with each other to form cohesive filamentous mats that maintain consistency and cohesion that is more similar to meats or textured plant proteins. A belt press can be particularly useful in producing large meaty slabs of dense cohesive biomass. These dense mats can be sliced, diced, chopped, molded, folded, extruded, or otherwise manipulated in a way known to a person with skill in the art to form slices, chunks, shreds, nuggets, or other particles and pieces.

In certain embodiments compositions comprising filamentous fungus biomass described herein are extruded using high temperature twin screw extrusion or other extrusion technology to form a material with a more conventional texture that is similar to textured vegetable proteins.

In certain embodiments the composition comprising filamentous fungus biomass is de-watered to remove moisture from the biomass. The material is optionally pasteurized in the substrate by using steam to heat the composition comprising filamentous fungus biomass (in the form of a slurry) to pasteurization temperatures (75-85° C.); the slurry is optionally released into a vibratory screen to de-water the material down to 75-95% water content; the composition comprising filamentous fungus biomass is then optionally pressed with a belt press or screw press or centrifuged to further reduce the water content; the material is then optionally shredded, sized, compacted, molded, otherwise formed, or combinations thereof; the material is then optionally added to a fluidized bed dryer for full dehydration. In particular embodiments, the material is de-watered to yield a water content for the particles as described below.

In certain embodiments the slurry described above, containing 1-8% biomass, is released into a belt press system. The material is simultaneously drained and pressed bringing the material down to 60-85% water content. The machine is adjusted to release a cake/slab at a thickness of 1 inch/2.54 cm. The 1 inch slab is continuously conveyed into a spindle and tine mechanical shredder. The shredder releases granular shreds in the size range of about 1 mm-about 20 mm. Shreds are continuously sieved using 2 mm and 12 mm sieves. Shreds released through the 2 mm sieve can be saved and de-hydrated separately or re-introduced to the initial slurry. Shreds released through the 12 mm sieve but not through the 2 mm sieve may be fed directly into a fluidized bed dryer for dehydration and optionally pasteurization. Shreds larger than 12 mm may be optionally conveyed back through the shredder for further size reduction. The dehydrated shreds between about 2 and about 12 mm are ready for use as an ingredient or to be further processed into ingredients described herein.

In certain embodiments the biomass slurry described hereinabove is de-watered down to 50-75% water content with methods known to a person with skill in the art of de-watering microbial biomass. The lower moisture biomass at 50-75% water content may then be fed through a dough chopping machine. Material is fed through a ¼ inch die and chopped into small particles intermittently by a rotating shear at the end of the die. This process may result in chunks of a consistent size of about (⅛ inch to ¼ inch) by (⅛ inch to ¼ inch) by (⅛ inch to ¼ inch).

Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine the organoleptic qualities, e.g., flavor, of the instant invention can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone. In the instant invention, it should be understood that there are any number of ways one of skill in the art could measure the sensory differences.

Embodiments of the present invention also include an edible filamentous fungal biomass composition made by the methods of the invention. Embodiments also include a composition which includes an edible filamentous fungus, wherein the filamentous fungus is Pleurotus spp., which was fermented in a media comprising at least 20 g/L date extract, and wherein the edible filamentous fungus was produced at a productivity of at least 20 g/L (dry weight).

Such produced edible filamentous fungal compositions can be used to create a number of food compositions, including, without limitation, spreads, pastes such as sweet (e.g. chocolate or fruit) pastes or savory pastes, prewhipped toppings, custards, coatings, peanut butter, frostings, cream filings, confectionery fillings, dairy alternative products, such as nondairy milks and nondairy cheeses and spreads, beverages and beverage bases, extruded and extruded/puffed products, meat imitations and extenders, baked goods and baking mixes, granola products, bar products, smoothies and juices, and soups and soup bases, all of which may comprise a composition comprising the filamentous fungal biomass according to the invention.

The edible filamentous fungal biomass can be used in a single or combination of ways. For example, the composition comprising the filamentous fungal biomass can be cooked at a temperature of less than 100° C. (e.g., 90° C., 80° C., 75° C., or 50° C., inclusive) for 1-60 minutes in dry or steam environments. The composition comprising the filamentous fungal biomass can be cooked at a temperature range of 100° C. to 200° C. (e.g. 100° C., 125° C., 150° C., or 200° C., inclusive) for 1-60 minutes in dry or steam environment. The composition comprising the filamentous fungal biomass can be cooked in a water bath at less than 100° C. for 1 minute to 120 minutes (e.g., 1, 2, 5, 10, 20, 40, 60, 80, 100, or 120 minutes, inclusive). In some embodiments, the fibrous mycelium mass can be stored. The composition comprising the filamentous fungal biomass can include additional ingredients. The composition comprising the filamentous fungal biomass can be cooked. The fibrous mycelium mass can be frozen at less than 0° C. under ambient or vacuum conditions, and/or refrigerated at less than 5° C. under ambient or vacuum conditions. The composition comprising the filamentous fungal biomass can be stored indefinitely in sealed container.

The invention includes methods to make food compositions, comprising providing a filamentous fungal biomass composition of the invention, providing an edible material, and mixing the filamentous fungal biomass compositions of the invention and edible material. The edible material can be, without limitation, a starch, a flour, a grain, a lipid, a colorant, a flavorant, an emulsifier, a sweetener, a vitamin, a mineral, a spice, a fiber, a protein powder, a plant protein powder, nutraceuticals, sterols, isoflavones, lignans, glucosamine, an herbal extract, xanthan, a gum, a hydrocolloid, a starch, a preservative, a legume product, a food particulate, and combinations thereof. A food particulate can include cereal grains, cereal flakes, crisped rice, puffed rice, oats, crisped oats, granola, wheat cereals, protein nuggets, texturized plant protein ingredients, flavored nuggets, cookie pieces, cracker pieces, pretzel pieces, crisps, soy grits, nuts, fruit pieces, corn cereals, seeds, popcorn, yogurt pieces, and combinations of any thereof.

Combinations with plant proteins and use of transglutaminase. In some embodiments, to form a food form, the composition comprising the filamentous fungal biomass may be combined with a plant protein, such as a pulse plant protein concentrate or isolate. In some embodiments, additional agents are added to improve the functionality of the combination, such as phosphates and/or one or more peptide cross linking enzyme, such as transglutaminase. In some embodiments, a process for making the combinations include a step of mixing the plant protein and the composition comprising the filamentous fungal biomass, heating the combination to between 40° C. and 65° C., and adding optional seasoning and an optional peptide cross linking enzyme such as transglutaminase, followed by further heating to a higher temperature (for example, between about 60° C. and 85° C., and adding an oil. The process may then include lowering the temperature to between 5° C. and 15° C. to form a precooked food form.

In other embodiments, the composition comprising the filamentous fungal biomass is mixed with additional proteins, such as pea protein, flavors and water using low shear. After mixing, the mass may be discharged into equipment common to the food industry used to produce shaped blocks by forming under pressure. Without being bound by theory, it may be that flow characteristics of the mix begin to introduce laminations which can be considered as textural pre-cursors for the final meat-like textural attributes achieved in the final product. The formed blocks may be raised to 90° C. by live steam. The blocks may then be chilled and frozen to approx −10° C. Freezing is carried out over ca. 30 minutes in order to achieve a controlled and slow rate of freezing relative to many other foods. Freezing may assist in the creation of the desirable meaty texture, with controlled growth of ice crystals helping to aggregate the hyphae to create fibrous bundles that can be described as meaty. The methods to produce a food composition can include the additional, optional steps of cooking, extruding, and/or puffing the food composition according to methods known in the art to form the food compositions comprising the filamentous fungal biomass compositions of the invention. In embodiments, the food composition comprises a combination of a edible filamentous fungal biomass of the invention and a plant protein.

In one embodiment, the food composition can include an alternative dairy product comprising a filamentous fungal biomass composition according to the invention. An alternative dairy product according to the invention includes, without limitation, products such as imitation skimmed milk, imitation whole milk, imitation cream, imitation cream filling, imitation fermented milk product, imitation cheese, imitation yogurt, imitation butter, imitation dairy spread, imitation butter milk, imitation acidified milk drink, imitation sour cream, imitation ice cream, imitation flavored milk drink, or an imitation dessert product based on milk components such as custard. Methods for producing alternative dairy products using alternative proteins, such as plant-based proteins as disclosed herein including nuts (almond, cashew), seeds (hemp), legumes (pea), rice, and soy are known in the art. These known methods for producing alternative dairy products using a plant-based protein can be adapted to use with a filamentous fungal biomass composition using art-known techniques.

The present invention can also include extruded and/or puffed products and/or cooked products comprising a filamentous fungal biomass composition of the invention. Extruded and/or puffed ready-to-eat breakfast cereals and snacks are known in the art. Extrusion processes are well known in the art and appropriate techniques can be determined by one of skill. These materials are formulated primarily with cereal grains and may contain flours from one or more cereal grains. The composition of the present invention contain flour from at least one cereal grain, preferably selected from corn and/or rice, or alternatively, wheat, rye, oats, barley, and mixtures thereof. The cereal grains used in the present invention are commercially available, and may be whole grain cereals, but more preferably are processed from crops according to conventional processes for forming refined cereal grains. The term “refined cereal grain” as used herein also includes derivatives of cereal grains such as starches, modified starches, flours, other derivatives of cereal grains commonly used in the art to form cereals, and any combination of such materials with other cereal grains.

The food product produced using the methods described herein can be in the form of crunchy curls, puffs, chips, crisps, crackers, wafers, flat breads, biscuits, crisp breads, protein inclusions, cones, cookies, flaked products, fortune cookies, etc. The food product can also be in the form of pasta, such as dry pasta or a ready-to-eat pasta. The product can be used as or in a snack food, cereal, or can be used as an ingredient in other foods such as a nutritional bar, breakfast bar, breakfast cereal, or candy. In a pasta, the one filamentous fungal biomass compositions may be, in a non-limiting example, be used in levels of about 10 g per 58 g serving (17%).

A food composition of the invention can also include a texturized protein, such as a texturized plant protein. Texturized plant protein comprising the filamentous fungal biomass compositions of the present invention include meat imitation products and methods for making meat imitation products comprising the filamentous fungal biomass compositions as disclosed within. The filamentous fungal biomass analog meat products can be produced with high moisture content using, for example, high moisture extrusion techniques to provide a product that simulates the fibrous structure of animal meat and has a desirable meat-like moisture, texture, mouthfeel, flavor and color. Methods for making such products using plant-based proteins such as pea protein, soy protein and the like are known in the art and such methods may be used in the instant invention. Texturization of protein is the development of a texture or a structure via a process involving heat, and/or shear and the addition of water. The texture or structure will be formed by protein fibers that will provide a meat-like appearance and perception when consumed. To make non-animal proteins palatable, texturization into fibrous meat analogs, for example, through extrusion processing has been an accepted approach. Due to its versatility, high productivity, energy efficiency and low cost, extrusion processing is widely used in the modern food industry. Extrusion processing is a multi-step and multifunctional operation, which leads to mixing, hydration, shear, homogenization, compression, deaeration. pasteurization or sterilization, stream alignment, shaping, expansion and/or fiber formation.

Meat analog products are made by high moisture extrusion processes. High-moisture extrusion processing can be used to create plant-based meat and seafood textures. During extrusion, proteins undergo thermal and mechanical stresses by heating of the barrel and shearing of the screws. As a result, protein structure is altered leading to the formation of soluble and/or insoluble aggregates. By attaching a long cooling die to the end of the extruder (as shown in the photograph on this page), proteins can be aligned in the flow direction forming an anisotropic protein network.

A wide range of final product characteristics can be achieved by altering process conditions during high-moisture extrusion processing. Process conditions in the screw section can be varied through independent process parameters, such as barrel temperature, screw speed, and configuration, whereas process conditions in the die section can be varied through cooling rate and die geometry. This improves the flexibility of the process to a significant extent. Extrusion is a multivariate complex process, and the sections are directly linked to each other. Any change in one section (e.g., cooling rate in die section) results in a change in process conditions in the other section (e.g., pressure and filling degree in screw section).

Several studies have been able to provide a better understanding of the effect of temperature and/or moisture content on molecular structure and physicochemical and final product properties for soy and pea proteins. Furthermore, thermal as well as mechanical treatment during extrusion processing affect proteins' molecular structure and product properties during extrusion processing.

High moisture extrusion processing can produce a more fibrous structure and meat-like texture of the resulting products. Appropriate pretreatment allows the use of a larger spectrum of proteins and other ingredients such as starches, fibers, and additives. By employing an additional processing step, the extruded mass is subjected to further treatment using forming units (additional plasticizing devices), where it is chilled, unified, textured, and/or molded into strips, patties, or other forms. High-moisture texturized proteins are usually processed and packaged in wet condition (pouches, cans, or frozen).

High-temperature and shear-induced structure formation of protein mixtures can also be achieved using a high-temperature conical shear cell. Wageningen University developed a larger-scale Couette shear device based on the concentric cylinder rheometer concept. The sample material is placed in the shearing zone space between the two cylinders; this space has a volume of ˜7 L and a 30 mm distance between the two cylinders. Both the inner and outer cylinders are heated by means of steam and cooled by means of air and/or water. The advantages of this new technology are the production of larger pieces of fibrous meat analogues with a simple, mild, and cost-effective technology.

The following examples further illustrate the invention but, of course, should not be construed as in any way limiting its scope.

### EXAMPLES

### Example 1

Testing for fungal strains capable of growing in date syrup. Date syrup contained 35% glucose. Yeast extract was product NFI 125 from Lallemand, Inc., a high protein yeast extract. 1 L of each medium was produced in a 2 L pyrex glass screw-cap bottle and sterilized for half an hour in an autoclave at 120-121° C. and once subsequently cooled to 55-60° C. was poured into 90 mm diameter Petri plates. All media contained 35 g/L date syrup (providing approximately 28° Brix in the final media), 5 g/L yeast extract, 15 g/L agar. Varying concentrations of magnesium sulfate heptahydrate and of potassium phosphate monobasic were tested; 1 g/L of each; 0 g/L of each; 0.5 g/L of each; 0.5 g/l potassium phosphate and 1 g/L of magnesium phosphate; and 1 g/l potassium phosphate and 0.5 g/L of magnesium phosphate.

The strains tested were Pleurotus eryngii, Morchella esculenta, Pholiota microspora and Lentinula edodes. All Petri plates were inoculated from agar slant cultures obtained from The Pennsylvania State University Mushroom Culture Collection, available from the College of Agriculture Sciences, Department of Plant Pathology and Environmental Microbiology, 117 Buckhout Laboratory, The Pennsylvania State University, University Park, Pennsylvania, USA 16802, and incubated at 26° C. The growth radius was recorded on days 5 & 8. P. eryngii exhibited growth of >25 mm (colony diameter), and M. esculenta exhibited growth of up to 30 mm (colony diameter.) On the other hand, P. microspore had maximum growth of up to 5 mm (colony diameter) and L. edodes, less than 5 mm. P. eryngii and M. esculenta exhibited much greater rates of growth than P. microspora or L. edodes which exhibited little growth. It was found in subsequent testing that other species of the Pleurotus genus grow well on date syrup-based media with a variety of nitrogen supplements. The addition of salts did not seem to affect growth (if anything the high phosphate content slightly inhibits growth of some strains) and that the date syrup and yeast extract are all that are needed for robust growth of the strains that grew well.

Further testing was performed as follows. Species tested include Agrocybe aegerita, Laetiporus sulphureus, Pleurotus djamor, Hypsizygus tesselatus, Laricifomes officinalis, Lignosus rhinocerus, Ophiocordyceps sphecocephala, Armillaria mellea, Ophiocordyceps sinensis, Termitomyces albuminosus, Hercium erinaceus, Agaricus blazei, Pleurotus spodoleucus, Pleurotus pulmonarius, Hypsizygus ulmarius and Volvariella volvacea. The agar contained 25 g/L date syrup, 1 g/L dibasic potassium phosphate, 0.5 g/L magnesium sulfate heptahydrate and 12 g/L agar. Four (4) nitrogen sources were tested, which were dibasic ammonium phosphate (media 1), urea (medium 2), sodium nitrate (medium 3) and monosodium glutamate (medium 4) at 3.5, 1.5, 3.5 & 3.5 g/L, respectively. All media were plated in triplicate and checked at days 5 & 8 for growth.

A table was developed binning each species in each media as either good, mediocre or bad regarding growth. It was found that the species/media combinations that enabled ‘good’ growth were A. aegerita in all media, V. volvacea in media 1, 3 & 4, P. pulmonarius in media 1, 2 & 4, P. spodoleucus in all media, T. albuminosus in media 1, 2 & 4, P. djamor in media 1, 2 & 4 and L. sulphureus in media 2, 3 & 4. ‘Mediocre’ growth was observed in A. aegerita in medium 3, V. volvacea in medium 2, P. pulmonarius in medium 3, H. ulmarius in all media, T. albuminosus in medium 3 and H. tesselatus in media 1 & 4. ‘Bad’ growth was observed in L. rhinocerus in all media, H. erincaceus in all media, A. blazei in all media, A. mellea in all media, L. officinalis in all media, O. sphecocephala in all media, H. tesselatus in media 2 & 3, L. sulphureus in media 1 and O. sinensis in all media.

### Example 2: Testing of N Sources—Shake Flask Testing

Date syrup is either obtained commercially or made by a method where dates were depitted, date flesh was washed thoroughly with a solution with 0.1% acetic acid and sodium bicarbonate, boiled, then diluted 1:1 to 1:5 with water to obtain a ‘syrup’ having approximately 70° Brix.

1 L baffled DeLong Erlenmeyer flasks were filled with 0.250 L of a medium consisting of 30 g/L date syrup (final Brix approximately 24°) and the following N sources as shown in Table 2, to identify the best sources of N for the growth of Pleurotus ostreatus, using the conditions as described in Example 1. Media was made up in RO water. The flasks were covered with a stainless-steel cap and sterilized in an autoclave on a liquid cycle that held the flasks at 120-121° C. for 1 hour. The flasks were carefully transferred to a clean HEPA laminar flowhood where they cooled for 18 hours. The flasks were subsequently inoculated with flask cultures in potato dextrose media of P. ostreatus, and grown for 7 days, filtered under aseptic conditions, resuspended in water at a 20× concentration, and each flask was inoculated with 0.5 mL. All flasks were placed on a shaker table at 120 rpm with a swing radius of 1″ at room temperature. Samples are taken at day 5 and day 7. Table 2 shows the listing of nitrogen sources that were tested, with the resultant biomass (biomass (g/L) at day 7. Biomass is measured by weight of material collected by filtration at the end of the culture.

TABLE 2

| Nitrogen Source | Biomass |
|---|---|
| Yeast extract | 23.54 |
| Cacao | 19.9 |
| Linseed | 19.76 |
| Tryptone | 19.18 |
| Wheatgrass | 16.64 |
| Chia | 15.83 |
| Lupin | 15.26 |
| Cottonseed | 15.12 |
| Hemp powder | 15.09 |
| Sodium nitrate (+ PP) | 14.42 |
| Pea protein | 14 |
| Coffee | 13.37 |
| Beer Spent | 13.29 |
| Soy flour | 13.25 |
| Ammonium Phosphate Dibasic (+ PP) | 12.66 |
| Goat Milk | 12.54 |
| Sunflower | 12.38 |
| Moong mogar | 12.26 |
| Cricket powder | 12.07 |
| Garbanzo flour | 11.89 |
| Green Lentil | 11.76 |
| Soybean | 11.42 |
| Rice Protein | 11.06 |
| Mung bean | 9.67 |
| MSG | 7.83 |
| Fava Bean | 6.78 |
| Kelp | 6.34 |
| Barley | 5.76 |
| Ammonium tartrate | 5.28 |
| Calcium nitrate | 2.06 |
| Ammonium nitrate (no PP) | 1.98 |
| Sodium nitrate (no PP) | 1.67 |
| Ammonium sulfate | 1.08 |
| Beer | 0.9 |
| Control | 0.86 |
| Urea | −0.26 |

### Example 3: Taguchi Study

Submerged culture conditions for optimal biomass production by Pleurotus ostreatus was optimized by Taguchi orthogonal array experimental design (DOE) methodology, adapting the methods shown in Prasad, K. et al., (2005) “Laccase production by Pleurotus ostreatus 1804: Optimization of submerged culture conditions by Taguchi DOE methodology,” Biochemical Engineering J. 24 17-26. Using the conditions of Example 1, a Taguchi design was carried out to evaluate the effect of the following factors: date syrup content, pea protein content, yeast extract content, magnesium sulfate, potassium phosphate. Results showed that biomass production in g/L was maximized at the following concentrations according to the DOE study: 31.3 g/L date syrup, 6 g/L yeast extract, 8 g/L pea protein, 0.5 g/L magnesium sulfate, 1 g/L potassium phosphate.

### Example 4: Initial Production Run Methodology, P. ostreatus

Inoculum preparation. This step aims to propagate the fungal biomass from glycerol stocks to 2 L flasks with a working volume of 0.5 L. Pleurotus ostreatus (obtained as described in Example 1) is the strain that was selected for this first process. This step allowed the reactivation of the fungi and acclimated it to further growth in a media containing date syrup as a carbon source. Media was produced, added to the flask, and then sterilized. P. ostreatus was then grown in an orbital incubator at 26° C. and 200 rpm. This stage is run in triplicate.

2 L Erlenmeyer flasks were filled with 0.5 L of a medium shown in Table 3. The flasks were wrapped with a sterilizable biowrap which was wrapped with autoclave tape 5-6 times (the taped biowrap should be easily taken off and put back on the flask without losing shape) and sterilized in an autoclave that held the flasks at 120-121° C. for 90 minutes. The flasks were carefully transferred to a clean HEPA laminar flowhood where they cooled for 18 hours. Each flask was subsequently inoculated with 2 cm² pieces of 60-day old P1 Petri plate cultures of P. ostreatus and placed on a shaker table at 120 rpm with a 1″ swing radius at 26° C. After a 48 hour incubation, pH was checked, and a microscope check was done to ensure the presence of mycelium and the culture was plated on LB media to ascertain the extent of any bacterial contamination.

TABLE 3
Inoculum preparation
Parameters and Ranges

| | | | Acceptable | |
|---|---|---|---|---|
| Parameter | Operating Range | | Range | |
| RO Water | 0.5 | L | ±0.05 | L |
| Date Syrup Charge | 12.75 | g | ±0 | g |
| Pea Protein Charge | 0 | g | ±0 | g |
| Yeast Extract | 4 | g | ±0 | g |
| Potassium Phosphate | 0.5 | g | ±0 | g |
| Magnesium Sulfate | 0.25 | g | ±0 | g |
| Antifoam | 0 | g | ±0 | g |
| Fermentation Air Sparge | N/A | | N/A | |
| Flow Rate | | | | |
| Media Sterilization | 123° | C. | ±2° | C. |
| Temperature | | | | |
| Media Sterilization Time | 0.5 | Hours | ±0.25 | Hours |
| Fermentation Temperature | 26° | C. | ±1° | C. |
| Fermentation Final pH | 5.5 | pH | ±0.2 | pH |

| | | |
|---|---|---|
| Fermentation Microscopy | No visible bacteria | — |
| Test | (100X) | |
| Petri film (24 h) | 0 | 0 |

S1 Fermentation: This fermentation was carried out in the S1 (25 L) bioreactor. The purpose of this step was to produce the inoculum for the main fermentation. Media components (Table 4) were charged to the vessel, along with RO water, and then batch sterilized. The sterilized media was inoculated with the culture produced in the inoculum preparation. The fermentation was continued until sugar (glucose) concentration, measured by YSI, fluctuated between 1 and 2 g/L. This step reached a biomass concentration close to 11 g/l to inoculate at 5-10% of the main fermentation. During this stage, samples of 100 mL were aseptically collected every 24 h to measure sugar content, pH and, biomass. Immediately after sample collection, sugar was quantified as a representation of fungal growth. If the sugar content was lower than 2 g/L, the main fermentation was started.

TABLE 4
S1 Fermentation (27 L)
Parameters and Ranges

| | | | Acceptable | |
|---|---|---|---|---|
| Parameter | Operating Range | | Range | |
| RO Water | 24.5 | L | ±0.5 | L |
| Date Syrup Charge | 625 | g | ±0 | g |
| Pea Protein Charge | 98 | g | ±0 | g |
| Yeast Extract | 49 | g | ±0 | g |
| Fermentation Air Sparge | 0.5 | VVM | ±0.5 | VVM |
| Flow Rate | | | | |
| Media Sterilization | 123° | C. | ±2° | C. |
| Temperature | | | | |
| Media Sterilization Time | 1.75 | Hours | ±0.25 | Hours |
| Pre-Inoculation Sterilized | 6 | pH | ±0.0 | pH |
| Media pH | | | | |
| Pre-Inoculation Sterilized | 20 | g/L | ±2 | g/L |
| Media Glucose | | | | |
| Fermentation Time | Contingent on | | ±3 | Hours |
| | Final Glucose | | | |
| Fermentation Temperature | 26° | C. | ±4° | C. |
| Fermentation Final pH | 5.5 | pH | ±1.0 | pH |

| | | |
|---|---|---|
| Fermentation Microscopy | No visible bacteria | — |
| Test | (100X) | |
| Petri film (24 h) | 0 | 0 |

Main Fermentation: The main fermentation to produce biomass was carried out in the S2 (250 L) bioreactors. This step aimed to produce the final intermediate that consists of fungal biomass rich in proteins. Media components were sterilized using the UHT continuous sterilization system, which directly fed into the fermenter S2. The sterilized media was inoculated with the culture produced in S1 fermentation at an inoculation ratio of 10%. The fermentation was sampled every 24 h during the first day and then every 12 h to make process decisions. After sampling, the sugar content was immediately estimated by YSI. The main fermentation was divided into two phases.

Phase 1. Initial Batch. Due to the inhibition of the fungal strain to high date syrup concentrations, the initial batch was carried out at an initial concentration of 30 g/L. In this first stage, the fungal biomass adapted to the new conditions and grew exponentially. The initial sugar was consumed, and then a fed-batch was carried out to enhance the fungal growth. Phase 2, a fed-batch, started when the sugar concentration is below 4 g/L. See Table 5 for conditions.

Phase 2. Fed-batch. This Phase 2 allowed reaching a higher fungal density. When the sugar (glucose) concentration of the previous stage was lower than 4 g/L, the fed batch was initiated, adding a certain amount of the feed as described in Table 6. Every 12 h, the reactor is sampled and, if the sugar concentration was lower than 4 g/L, an addition of sterilized concentrated media was done. The fed media were produced in S1 after inoculating S2 and washing the tank. The media components, as described in Table 6, were charged to the vessel, along with RO water, and then batch sterilized. The feeding strategy carried out is described in Table 7.

TABLE 5
S2 Main Fermentation (320 L)
Parameters and Ranges

| | | | Acceptable | |
|---|---|---|---|---|
| Parameter | Operating Range | | Range | |
| RO Water | 190 | L | ±5 | L |
| Date Syrup Charge | 6.260 kg | (6260 g) | ±0 | Kg |
| Pea Protein Charge | 1.600 kg | (1,600 g) | ±0 | Kg |
| Yeast Extract | 1.200 kg | (1,200 g) | ±0 | Kg |
| Potassium Phosphate | 0.200 kg | (200 g) | ±0 | Kg |
| Magnesium Sulfate | 0.100 kg | (100 g) | ±0 | Kg |
| Antifoam | 40 | g | ±0 | Kg |
| Fermentation Air Sparge | 0.2 | VVM | ±0.5 | VVM |
| Flow Rate Start | | | | |
| Media Sterilization | 123° | C. | ±2° | C. |
| Temperature | | | | |
| Media Sterilization Time | 1.75 | Hours | ±0.25 | Hours |
| Pre-Inoculation Sterilized | 6 | pH | ±1.0 | pH |
| Media pH | | | | |
| Pre-Inoculation Sterilized | >13 | g/L | ±0.5 | g/L |
| Media Glucose | | | | |
| Agitation Rate | From 60 to 90% | | ±0 | rpm |
| | of capacity | | | |
| Fermentation Time | Once 25 L of Feed media | | ±3 | Hours |
| | is completed and, | | | |
| | Glucose is <1 g/L or | | | |
| | rheology of media makes | | | |
| | fermentation inoperable. | | | |
| Fermentation Temperature | 26° | C. | ±4° | C. |
| Fermentation final pH | 5.5 | pH | ±1.0 | pH |

| | | |
|---|---|---|
| Fermentation Microscopy | No visible bacteria | — |
| Test | (100X) | |
| Petri film (24 h) | 0 | 0 |

TABLE 6
Feed for the second stage of Main Fermentation
Parameters and Ranges

| | | | Acceptable | |
|---|---|---|---|---|
| Parameter | Operating Range | | Range | |
| RO Water | 20 | L | ±5 | L |
| Date Syrup Charge | 6.17 kg | (6,170 g) | ±0 | kg |
| Pea Protein Charge | 1.588 kg | (1,588 g) | ±0 | kg |
| Yeast Extract | 1.190 kg | (1,190 g) | ±0 | kg |
| Antifoam | 0.1 kg | (100 g) | ±10 | g |
| Media Sterilization | 123° | C. | ±2° | C. |
| Temperature | | | | |
| Media Sterilization Time | 1.75 | Hours | ±0.25 | Hours |
| Pre-Inoculation Sterilized | 6 | pH | ±1.0 | pH |
| Media pH | | | | |
| Agitation Rate | From 60 to 90% | | ±10% | |
| | of capacity | | | |
| Temperature | 26° | C. | ±3° | C. |

TABLE 7
Feed strategy
Parameters and Ranges

| | |
|---|---|
| Sampling | Every 12 h |
| Parameters to test | pH, microbiology test, biomass, |
| | and sugar concentration. |
| Sugar concentration | YSI analysis |
| Acceptable sugar concentration | >4 g/L |

If sugar concentration is lower than 4 g/L, add feed as indicated:

| | | |
|---|---|---|
| Parameter | Operating Range | Acceptable Range |

| | | | | |
|---|---|---|---|---|
| 4 g/L | 1.5 | L | ±5 | L |
| 3 g/L | 3 | L | ±0 | kg |
| 2 g/L | 4.5 | L | ±0 | kg |
| 1 g/L | 6 | L | ±0 | kg |
| 0 g/L | 7.5 | L | ±0 | kg |

Heat Treatment: The purpose of this step was to complete the fermentation process by introducing steam and raising the temperature of the Main Fermenter to a temperature of 50° C. The Main Fermenter was held at 50° C. for 2.5±0.5 hours to inactivate the biomass before being transferred into the filtration stage.

Filtration. This step concentrated the biomass before drying it by eliminating the supernatant and the water excess. The content of the S2 tank was discharged in pre-bought cloth bag filters. The supernatant was directed to the wastewater treatment plant. The water excess of the captured biomass was removed by compressing the bags.

Yields from the fermentation of g/L/day and g/L total. After 9 days in the Main Fermenter, yields of biomass were 18 g/L, and a rate of 2.0 g/L/day.

Drying. The fungal biomass was spray-dried after filtration. After this stage, several tests are made with the biomass such as extrusion and flavor testing.

The culture was finally spray dried and tasted. The final product was noted to have a mild aroma with a mild umami taste at concentrations up to 10% w/v slurry in water.

Analytical testing showed protein at 46.56%, nitrogen 7.45%, protein factor 6.25, using AOAC 990.03, AOAC 992.15 methods. Amino acid analysis showed alanine 2.43%, arginine 2.65%, aspartic acid 4.35%, glutamic acid 5.81%, glycine 1.89%, histidine 0.97%, isoleucine 2.01%, leucine 3.24%, phenylalanine 2.02%, proline 1.86%, serine 1.99%, threonine 1.93%, total lysine 2.52%, tyrosine 1.25%, valine 2.67% (AOAC 982.30 mod.) cystine 0.43% and methionine, 0.55% (AOAC 994.12 mod.), and tryptophan 0.54% (AOAC 988.15 mod.) PDCAAS IS 84 for adults and 72 for infants, per Protein quality evaluation. Report of an FAO/WHO Expert Consultation. FAO food and nutrition paper no. 51. Food and Agriculture Organization of the United Nations. Rome, 1991. (1991) and Dabbour, I R; Takruri, H R. “Protein digestibility using corrected amino acid score method (PDCAAS) of four types of mushrooms grown in Jordan”, Plant Foods for Human Nutrition. 2002, Winter, 57(1), pp 13-24.

### Example 5: Flask Experiment (1 L) Flask. Testing of N Sources

Date syrup is either obtained commercially or made by a method where dates were depitted, date flesh was washed thoroughly with a solution with 0.1% acetic acid and sodium bicarbonate, boiled, then diluted 1:1 to 1:5 with water until obtaining a ‘syrup’. All media contained date syrup (e.g., 35 g/L providing approximately 28° Brix in the final media),

Per Example 1, media was made up in RO water. Media (C-source, and N-source) was prepared and sterilized in different containers to minimize color changes to Maillard reactions. The flasks were covered with a stainless-steel cap and sterilized in an autoclave on a liquid cycle that held the flasks at 120-121° C. for 1 hour. The flasks were carefully transferred to a clean HEPA laminar flowhood where they cooled for 18 hours. The flasks were subsequently inoculated with flask cultures in potato dextrose media of P. eryngii, and grown for 7 days, filtered under aseptic conditions, resuspended in water at a 20× concentration, and each flask was inoculated with 0.5 mL. All flasks were placed on a shaker table at 120 rpm with a swing radius of 1″ at room temperature.

To initiate the experiment, 1 L baffled DeLong Erlenmeyer flasks were filled with 0.250 L of a medium consisting of 25 g/L date syrup, 0.5 g/L MgSO₄, 1 g/L KH₂PO₄ and the following N sources, equivalent to 0.7 g/L of nitrogen to work at a C:N ratio of 10. The compounds tested were 1. ammonium chloride (2.67 g/L), 2. ammonium phosphate dibasic (3.3 g/L), 3. ammonium sulfate (3.3 g/L), 3. beer spent grain (10 g/L), 4. pea protein (5.5 g/L), 5. peptone from pea (4.827 g/L), 6. tryptone (5.83 g/L), 7. urea (1.5 g/L) and 8. yeast extract (5.36 g/L). A control without any nitrogen source was also added., to identify the best sources of N for the growth of Pleurotus eryngii (obtained from The Pennsylvania State University Mushroom Culture Collection, available from the College of Agriculture Sciences, Department of Plant Pathology and Environmental Microbiology, 117 Buckhout Laboratory, The Pennsylvania State University, University Park, Pennsylvania, USA 16802). In one container, the solution containing date syrup and salts was heat sterilized, nitrogen solutions were filter sterilized if possible, and then added to the bottle containing the carbon source. Due to the hydrophobicity of pea protein and beer spent, solutions were heat sterilized. pH was adjusted to 5.5 in all cases using NaCl or KOH.

Flasks were inoculated with 1 mL of a solution with P. eryngii and grown at 26° C. and 120 rpm on a shaker table with a swing radius of 1 inch which had been briefly homogenized immediately prior to inoculation. Parameters for a mature inoculum (ready for use): growth for 10 to 15 days, final glucose concentration 1 to 4 g/L, pH 6-7. After days 4, 7, and 10 of shake flask culture, one or two flasks were removed to be analyzed. Culture broth was filtered using coffee filters and then the separated biomass was freeze dried and then, weighed. Protein content was estimated at day 10 using a modified Bradford analysis.

Results show that the morphology of biomass at day 4 and day 10, the fungal morphology was mostly mycelial pellets with diameters around 1 mm. The highest biomass was obtained from use of yeast extract is used as the nitrogen source, with a biomass at 10 days of fermentation of around 11 g/L. After 10 days, 8 g/L biomass were produced when urea, pea protein, peptone from pea and tryptone were used as the nitrogen source. Urea and pea protein were the least expensive nitrogen sources tested. On the other hand, inorganic nitrogen sources were not easily assimilated by P. eryngii, achieving less than 1.5 g biomass after ten days.

### Example 6. Shake Flask Experiment Testing Additional N Sources

Using the methods described in Example 5, eight nitrogen compounds were tested on the growth of P. eryngii. In this case, the nitrogen sources were selected based on its cost per metric ton (Table 8). Media were prepared using 30 g/L of date syrup, 0.5 g/L of MgSO₄ and 1 g/L of KH₂PO₄ and the amounts indicated in Table 8 that are equivalent to a nitrogen content of 0.7 g.

TABLE 8
Concentration of nitrogen compounds used and its costs.

| | Concentration | Cost | Amount of N (g | Cost per |
|---|---|---|---|---|
| Compound | (g/L) | ($/kg) | N/g compound) | kg of N |
| Rice Protein | 6.03 | 2.6 | 0.13 | 20.43 |
| Linseed (flax) | 11.67 | 0.3 | 0.06 | 5.00 |
| meal | | | | |
| Monosodium | 7.4 | 0.5 | 0.09 | 5.29 |
| glutamate | | | | |
| Cottonseed meal | 11.67 | 0.54 | 0.06 | 9.00 |
| Soybean meal | 10 | 0.12 | 0.07 | 1.71 |
| Corn gluten meal | 7 | 0.75 | 0.1 | 7.50 |
| Corn steep | 10.7 | 0.4 | 0.07 | 10 |
| powder | | | | |
| Calcium nitrate | 2 | 0.2 | 0.17 | 1.17 |

The results show that calcium nitrate did not favor biomass growth. In contrast, soybean meal, corn steep powder, and cotton seed meal were useful for supporting biomass growth.

### Example 7. Further Evaluation of Urea as an N Source

After analyzing the cost of the nitrogen sources, evaluating the cost of scaling up the process and identifying the highest biomass yields, an experiment to evaluate the effect of urea in the growth of P. eryngii was designed. Using conditions as described in Example 5, use of urea as the sole nitrogen source was investigated using a media containing different concentrations of the compound ranging from 0.5 to 4 g/L. Media contained 30 g/L of date syrup, 0.5 g/L of MgSO₄ and 1 g/L of KH₂PO₄ and urea. Media was autoclaved, pH was adjusted with citric acid after sterilization and then, inoculated with a 1 mL of a blended P. eryngii solution as described previously. Four flasks of each condition were run at 26° C. and 120 rpm. It was observed that after 8 days, 10 g/L of biomass was obtained when a concentration of 2 g/L of urea was used. Due the low cost of urea, and the ability of P. eryngii to grow in the compound further studies were carried out using a combination of urea and another nitrogen source.

### Example 8. Shake Flask Experiment on Combination of Pea Protein and Urea as N Sources

After analyzing the cost of the nitrogen sources, evaluating the cost of scaling up the process and identifying the highest biomass yields, an experiment to evaluate the effect of a combination media using both pea protein and urea in the growth of P. eryngii was designed.

Using conditions as described in Example 5, use of combinations of pea protein and urea as the nitrogen source was investigated using a media containing different concentrations of the compound ranging from 0.5 to 4 g/L. Media contained 30 g/L of date syrup, 0.5 g/L of MgSO₄ and 1 g/L of KH₂PO₄ and urea. Media was autoclaved, pH was adjusted with citric acid after sterilization and then, inoculated with a 1 mL of a blended P. eryngii solution as described previously. Four flasks of each condition were run at 26° C. and 120 rpm.

It was observed that after 8 days, 10 g/L of biomass was obtained when a concentration of 2 g/L of urea was used. Due the low cost of urea, and the ability of P. eryngii to grow in the compound further studies were carried out using a combination of urea and another nitrogen source. Table 9 shows the combinations of urea and pea protein used.

TABLE 9
Media composition of a mixture of urea and pea protein.

| | | Pea |
|---|---|---|
| | Urea | Protein |
| 1 | 1 | 4 |
| 2 | 1 | 6 |
| 3 | 1 | 8 |
| 4 | 1 | 10 |
| 5 | 2 | 4 |
| 6 | 2 | 6 |
| 7 | 2 | 8 |
| 8 | 2 | 10 |
| 9 | 3 | 4 |
| 10 | 3 | 6 |
| 11 | 3 | 8 |
| 12 | 3 | 10 |
| PP4 | 0 | 4 |
| PP6 | 0 | 6 |
| PP8 | 0 | 8 |
| PP10 | 0 | 10 |

Glucose concentrations were measured using a glucose refractometer (YSI Inc., Yellow Springs, Ohio). Biomass production was maximized at the following concentrations according to the DOE study: the use of two different nitrogen sources had a positive effect in biomass growth at day 5. Particularly, the highest concentrations were obtained using a media comprising 2 g/L of urea and 10 g/L of pea protein.

At day 7, two maximum peaks are evident. One that coincided to the last condition (i.e., 10 g/L pea protein, 2 g/L of urea) and an additional one that is at 10 g/L of pea protein. After analyzing results related to the carbon content as glucose concentration, in general, sugar was consumed faster when urea was present.

### Example 9: Taguchi Study. Shake Flask

Submerged culture conditions for optimal biomass production using media by Pleurotus eryngii was optimized by Taguchi orthogonal array experimental design (DOE) methodology, per Example 3. Taguchi design was carried out to in the following conditions at 2 g/L urea and 10 g/L pea protein, using the parameters shown in Table 10, using the conditions as provided in Example 5, to optimize the levels of salts and pH for production.

TABLE 10
Media composition of a mixture of urea and pea protein

| Media ID | Date syrup | MgSO₄ | KH₂PO₄ | pH |
|---|---|---|---|---|
| 1 | 25 | 0.5 | 1 | 4.7-5 |
| 2 | 25 | 1 | 2 | 5.4-5.7 |
| 3 | 25 | 2 | 4 | 6-6.3 |
| 4 | 30 | 0.5 | 2 | 6-6.3 |
| 5 | 30 | 1 | 4 | 4.7-5 |
| 6 | 30 | 2 | 1 | 5.4-5.7 |
| 7 | 35 | 0.5 | 4 | 5.4-5.7 |
| 8 | 35 | 1 | 1 | 6-6.3 |
| 9 | 35 | 2 | 2 | 4.7-5 |

Carbon source was sterilized in a different container than nitrogen sources, after sterilization media was prepared by mixing both containers. pH was adjusted under aseptic conditions using pre-sterilized citric acid. Media was inoculated using 1 mL of a P. eryngii solution and then placed at 120 rpm and 26° C. Biomass was quantified by duplicate at day 5 and 7. Data was analyzed using MiniTab software. According to the analysis, the biomass that can be obtained when the best conditions are used was predicted to be 13.11 g/L and standard deviation of 0.957. According to this analysis, best conditions are date syrup at 35 g/L, MgSO₄ at 2 g/L, KH₂PO₄ at 4 g/L and pH at 5.

### Example 10. Shake Flask Study on Growth in Glucose or Fructose

Date syrup is comprised essentially of sugars. It is known that 77.4% of date syrup are carbohydrates. From there 73.18% is glucose, 33.26% is fructose and the rest are other sugars like lactose, maltose, and sucrose. For that reason, and to assure the complete degradation of sugars, the growth of P. eryngii in the presence of glucose and fructose was evaluated to ensure that the organism is capable of utilizing both fructose and glucose as carbon sources. Using the methods described in Example 5, media were prepared using 30 g/L of either fructose or glucose, with each containing 2 g/L urea, 0.5 g/L of MgSO₄ and 1 g/L of KH₂PO₄. Biomass was grown for seven days and harvested as discussed in Example 5, and the biomass measured. The data from the experiment showed that growth on the glucose source yielded approximately 8 g/L and growth on fructose yielded approximately 6 g/L, indicating that this organism can use fructose and thus can utilize the fructose in date syrup for growth.

### Example 11. Shake Flask Study on Evaluating Different Counterion for Sulfates

To evaluate the effect of different sources of sulfates on biomass productivity using Mg, Ca, and Fe sulfates, using 250 mL shake flasks. Using the methods described in Example 5, media were prepared using 35 g/L of date syrup, 2 g/L urea, and 1 g/L of KH₂PO₄, together with. Biomass was grown for five days and seven days and harvested as discussed in Example 5, and the biomass measured. The amounts of each sulfate were adjusted to provide equimolar amounts of sulfate; thus 125 mg of MgSO₄, 140 mg FeSO₄, and 86 mg of CaSO₄ were used. The data from the experiment showed that growth using MgSO₄ and CaSO₄ yielded approximately 10 g/L at five days and 18 g/L at 7 days, whereas growth at seven days in FeSO₄ was approximately 13 g/L, or significantly less, indicating that this organism grows best on magnesium sulfate or calcium sulfate.

### Example 12

This is a shake flask study on evaluating how organism performs when carbon source is changed from glucose to fructose during the fermentation. The aim of this experiment is to confirm that the organism can switch to utilizing fructose after using glucose as a carbon source.

Using the methods described in Example 5, six flasks were prepared using 15 g/L of glucose, 2 g/L urea, 2 g/L of KH₂PO₄, and 2 g/L MgSO₄. Biomass was grown for 7 days, and when glucose is depleted (by glucose refractometer measurement), three flasks are sacrificed and the amount of biomass measured. To the remaining flasks, 15 g/L fructose is added, and the flasks grown for an additional 5 days. The data from the experiment showed that biomass accumulation is higher when glucose rather than fructose is used as carbon source. When fructose is added to biomass that has already consumed glucose, the productivity is lower and production yield is 0.26 g biomass per g added sugar, instead of 0.8 when using glucose. Thus, while the organism can consume fructose, this sugar is not as efficient at allowing the biomass to accumulate.

### Example 13

Shake flask study on evaluating how organism performs with different levels of pea protein and urea using conditions as outlined in Example 5. Different ratios of pea protein and urea were tested, using 35 g/L date syrup and 2 g/L of KH₂PO₄, and 2 g/L MgSO₄. Levels of pea protein tested were 3, 5, 7 and 10 g/L and 0, 1, 2, and 3 g/L of urea. At day 5 of the fermentation, it was found that the combination of 7 g pea protein and 3 g urea per liter provided the best productivity.

### Example 14. Bioreactor Methods

Reactors (7 L, with a 4 L working volume) MINIFORS, Infors (Annapolis Junction, MD); BIOFLO 120, Eppendorf, Enfield, CT, or CELLIGEN 310, New Brunswick (Eppendorf, Enfield, CT). Working volume is 7 L were run using art-known procedures as follows. Glucose was monitored daily via YSI, and once the glucose levels had reached 2 g/L, or close to, 1000 mL of concentrated media was added at 0.75 mass content of the original reactor conditions. Glucose was again monitored until depletion and an additional fermentation time of a day was to allow the fungi to deplete residual free fructose in the media.

Prepare bioreactors by setting up all attachments and preparing inoculation, antifoam, date syrup, and exhaust bottles. Acid and base (if used) will be filtered into a sterile container instead of being sterilized in an autoclave.

To set up the bioreactor, attach two Rushton turbines to each agitator shaft. Distance between the two impellers should be the diameter of the Rushton turbine. Clearance of the lowest Rushton turbine from the bottom of the glass vessel should be 2.3″ (5.8 cm).

Calibrate pH probe and set aside along with the DO probe to avoid accidental damage while assembling the reactor. Measure out media using the compositions above in the Materials section. The nitrogen sources—pea protein and urea—can be transferred directly to the reactors. Fill the glass vessels to 3.2 L using RO water and stir all solids into solution.

The carbon source—date syrup—and salts should be mixed in separate 1-L bottles and sterilized separately to avoid potential Maillard reactions. Fill to 400 mL with RO water and shake to dissolve all solubles into solution. Verify that all attachments are clamped tight, except for exhaust, and ensure that there are no loose components. All air filter lines should be unobstructed. Attach pH and DO probes to reactor headplates.

Insert bioreactors and empty inoculation bottles into autoclave and run for 60 minutes.

Remove reactors out of autoclave and attach to control panels in the pilot lab. Cooling loops, spargers, and agitators should be attached and started first to mitigate contamination by creating positive pressure. Let reactors cool to 26° C. and attach remaining cables or tubes. Prior to attaching DO probe to cable, zero the sensor to 0% DO through the control panel Attach a sterile 10-mL syringe containing sterilized citric acid and another containing sterilized antifoam. Use these to manually adjust pH or eliminate foaming as needed.

Add date syrup media to each bioreactor and take post-sterilization samples from each reactor to check glucose concentrations. DO should be re-standardized to 100% once agitation and air flow has been left to run overnight.

Transfer 400 mL of inoculum to each sterile inoculum bottle in the culture room. Connect the inoculum bottle to the reactor using the quick-connect port and aseptically transfer into the reactor using an external pump. Inoculum is prepared as described in Example 5.

Take samples from each reactor to verify offline pH and glucose concentrations. If online pH has drifted, re-ZERO the pH to the offline reading.

If pH is above 5.0, manually add citric acid. If foaming, manually add antifoam.

Take daily samples to monitor glucose. Follow the additional protocol below to withdraw samples and prepare for biomass measurements.

Allow reactors to ferment until glucose concentrations approach 0 g/L. At this point, supplement each reactor with additional sterilized media at 75% concentration until glucose depletes again.

Leave the reactors to ferment one extra day after glucose has depleted to reduce fructose concentration.

Begin harvesting all reactors and filtering biomass through Miracloth. Some supernatant should be saved and filtered through a 2-μm syringe filter to be used for HPLC analysis.

Plate out Miracloth-filtered biomass on metal trays and freeze them to prepare for freeze drying. After these are frozen, place in the freeze dryer and run the unit. Characterize dried product and analyze data.

### Example 15

Using the methods of Example 14, P.eryngii was grown in a 7 L bioreactor using media consisting of 2 g/L of urea, 10 g/L of pea protein, 35 g/L of date syrup, 1 g/L KH₂PO₄ and 0.5 g/L of MgSO₄. Reactor was operated in batch and at agitation of 150 rpm. The results showed that for a duration of fermentation of 7 days, a yield of 19 g/L, and a productivity of 2.7 g/L/day was obtained.

### Example 16

Using the methods of Example 14, P.ostreatus was grown in a 7 L bioreactor using 25 g/L of date syrup, 1 g/L KH₂PO₄ and 0.5 g/L of MgSO₄, 10 g/L yeast extract, 120 agitation was kept at 120 rpm (approximately 0.8 m/s impeller tip speed), 0.75 VVM, pH 5-6. The results showed for a duration of fermentation of 7 days, a yield of 15 g/L, and a protein content of 46% was achieved.

### Example 17. Testing Partial Substitution of Pea Protein for Yeast Extract

Using the methods of Example 14, P.ostreatus was grown in a 7 L bioreactor using 30 g/L date syrup, 1 g/L KH₂PO₄ and 0.5 g/L of MgSO₄ and for condition B3, 4 g/L pea protein and 6 g/L yeast extract, and for condition B4, 10.7 g/L yeast extract.

Agitation was kept at 120 rpm (approximately 0.8 m/s impeller tip speed), 0.75 VVM, pH 5-6. A feed was done after day 6, where 200 mL of 20 g/L date syrup was added from day 6 to day 11. Fermentation was finished at day 12. Results: B3: 128.3 g of biomass in a final volume of 4.7 L, final biomass was 27.3 g/L and a productivity of 2.12 g/L/day. For B4: 98.3 g total in 4.75 L, final biomass of 20.7 g/L and productivity of 1.62 g/L/day. Results: Pea protein presence increased productivity.

### Example 18. Date Slurry

Using the methods of Example 14, P.ostreatus was grown in a 7 L bioreactor using a date slurry prepared as follows: dates were depitted, date flesh was washed thoroughly with a solution with 0.1% acetic acid and sodium bicarbonate, boiled, then diluted 1:1 to 1:5 with water until obtaining a ‘syrup’ having approximately 70° Brix. 25 g/L of date syrup, 1 g/L KH₂PO₄ and 0.5 g/L of MgSO₄, 10 g/L yeast extract, 2 g/L urea; agitation was kept at 120 rpm, (approximately 0.8 m/s impeller tip speed), 0.75 VVM, pH 5-6. At days 7 and 8, two additions of fresh media were performed of 150 mL and 200 mL. Final dry mass of reactor was 229.46 g after freeze-drying, with a productivity of 3.45 g/L/day, with a final biomass of 31 g/L. This result showed that the organism could be grown on date slurry.

### Example 19. Higher Agitation Methods

Using the methods of Example 14, P.eryngii was grown in a 7 L bioreactor using higher agitation levels, increased from 120-200 rpm (approximately 0.8 m/s to 1.33 m/s impeller tip speed), to 400 rpm (approximately 2.66 m/s impeller tip speed), (at 0.75 VVM). Media was 2 g/L of urea, 10 g/L of pea protein, 35 g/L of date syrup, 1 g/L KH₂PO₄ and 0.5 g/L of MgSO₄. Due to the growth form of the mycelia, e.g., mycelial pellets, it was predicted that higher agitation rates would provide too much shear stress to the organism and its mycelial growth habits. However, the results showed that higher agitation rates (approximately 2.66 m/s impeller tip speed), lead to increased productivity. Increased agitation had a positive effect with productivity increase observed to 5.6 g/L/day from levels observed from levels of about 3 g/L/day.

### Example 20. Testing Different Concentrations of Potassium Phosphate

Using the methods of Example 14, P.eryngii was grown in a 7 L bioreactor to evaluate the effect of potassium phosphate monobasic concentrations on the final biomass, protein content, and protein quality from fermentation of P. eryngii in pea protein and date syrup. Media was 2 g/L of urea, 10 g/L of pea protein, 35 g/L of date syrup, 2 g/L or 1 g/L KH₂PO₄ and 0.5 g/L of MgSO₄. Agitation was kept at 400 rpm, (approximately 2.66 m/s impeller tip speed) 0.775 VVM, pH 5-6.

At 2 g/L of KH₂PO₄ the yield was 40.29 g/L of biomass and productivity was 5.76 g/l/d. Protein accumulation was 39.44%. At 1 g/L of KH₂PO₄ the yield was 42.29 g/L of biomass and productivity was 6.05 g/l/d. Protein accumulation was 36.25%. Results show a total protein analysis using the Kjeldahl method indicated that the culture grown with 2 g/L KH₂PO₄ produced the highest protein content at 39.44%. For the culture grown in 1 g/L KH₂PO₄, final protein content was 36.25%. Overall the experiment showed that while there was a slight advantage to 1 g/L of KH₂PO₄, however, the differences were not significant.

### Example 21. Different Feeding Strategies

Using the methods of Example 14, P.eryngii was grown in a 7 L bioreactor to evaluate different feeding strategies on productivity. Four different strategies were used in this experiment. A no feeding (control), was compared to three different feeding strategies: 1) feeding one time once glucose reaches zero, 2) feeding once daily to maintain >2 g/L of glucose concentration and 3) feeding once daily to maintain >4 g/L Media was 2 g/L of urea, 10 g/L of pea protein, 35 g/L of date syrup, 2 g/L or 1 g/L KH₂PO₄ and 0.5 g/L of MgSO₄. Agitation was kept at 400 rpm, (approximately 2.66 m/s impeller tip speed), 0.775 VVM, pH 5-6. Results from this experiment suggest that fed-batch operation is the optimal mode for yield, productivity, and protein content. Batch fermentation produced a yield of 32 g/L, with a productivity of 3.6 g/L/day. The fed batch yielded 44 g/L, with a productivity of 3/2 g/L/day. Feeding multiple times (once per day) to keep the glucose concentration at 2 g/L resulted in 24 g/L, with a productivity of 1.5 g/L/day and feeling multiple times (once per day) to keep the glucose concentration at 4 g/L, resulted in 43 g/L, with productivity at 3.9 g/L/day. Results showed that fed-batch produces better results, but there was no additional benefit to multiple feedings.

### Example 22. Addition of Thiamine

Using the methods of Example 14, P.eryngii is grown in a 7 L bioreactor to evaluate addition of thiamine to the media to increase productivity. Media is 0.25 mg/L thiamine, 2 g/L of urea, 10 g/L of pea protein, 35 g/L of date syrup, 2 g/L KH₂PO₄ and 0.5 g/L of MgSO₄. Agitation was kept at 400 rpm, (approximately 2.66 m/s impeller tip speed), 0.775 VVM, pH 5-6. This experiment is run as fed-batch as shown in Example 21. Results from this experiment suggest that thiamine increases productivity levels.

### Example 23. Addition of Oxygen

Using the methods of Example 14, P.eryngii is grown in a 7 L bioreactor to evaluate addition of pure oxygen to the airflow during the fermenter run to increase productivity. Oxygen is controlled to result in 20 to 40% of dissolved oxygen in the media, the control is performed by a cascade increase in the agitation to 700 rpm and the oxygen is injected to a maximum of 1 L/min. Media is 0.25 mg/L thiamine, 2 g/L of urea, 10 g/L of pea protein, 35 g/L of date syrup, 2 g/L KH₂PO₄ and 0.5 g/L of MgSO₄. Agitation was kept at 400 rpm (approximately 2.66 m/s impeller tip speed), 0.775 VVM, pH 5-6. This experiment is run as fed-batch as shown in Example 21. Results from this experiment suggest that oxygen addition increases productivity levels.

### Example 24

Test effect of autoclaving versus filter sterilizing urea when inoculating seed flasks from stock grainspawn to determine whether potential production of ammonia from autoclaved urea inhibited production rates.

This experiment was run as described in Example 5. This experiment involved preparing 300 mL working volume media in 1 L baffled, DeLong Erlenmeyer flasks for 5 various experimental conditions, all done in triplicate. The experiment tested date syrup at 52.5 g/L, urea at 3 g/L. The date syrup, urea and salts used in the experiment were all sterilized as separate concentrates, which were added to the flasks post-sterilization to create the 300 mL working volume. Conditions 1 & 2 contained autoclaved and filter sterilized urea, respectively. Conditions 3 & 4 were prepared similarly as 1 & 2 but also contained 0.5 g/L magnesium sulfate heptahydrate and 1.5 g/L potassium phosphate monobasic. A 5th condition with no salt and autoclaved urea at 35 g/L date syrup and 2 g/L urea was also included to analyze the results of conditions 1-4 with a lower concentration date syrup medium. The flasks were inoculated with ˜1.7 g of a dried, blended grainspawn that had been stored at 4° C. for approximately 1 month of Pleurotus eryngii and incubated on a shaker table at 26° C. and 120 RPM. One (1), 5 mL sample was taken from the flasks every 3-4 days to analyze for glucose.

It was found that autoclaving versus filter sterilizing the urea had no effect on performance (i.e. glucose consumption), though the pH of the urea stock culture increased dramatically post-sterilization, presumably due to ammonia formation. After approximately 33 days, the flasks were filtered and the retentate washed and desiccated for dry cell weight (i.e. biomass) determination. Conditions 1˜4 all developed ˜17-19 g/L of biomass whereas condition 5 developed 12 g/L biomass.

### Example 25

Test 1% vs. 5% inoculation ratios at 105, 52.5 and 35 g/L date syrup recipes, shake flask.

This experiment was run as described in Example 5, using flasks from Example 24 as seed culture. This experiment tested submerged culture conditions for optimal biomass production using higher date syrup levels, urea, inoculation ratio, as well as use of seed flask original concentration of date syrup; all conditions in triplicate; by Pleurotus eryngii. Date syrup concentrations were tested at 35, 52.5, and 105 g/L; urea was tested at 2, 3, and 6 g/L, and inoculation ratios were tested at 1% and 5%.

Results show that the yield of biomass is approximately 30 to 32 g/L when grown at 105 g/L whereas for 52.5 g/L, yield is approximately 15 g/L. This example showed that P. eryngii grew well in up to about 105 g/L date syrup when nitrogen source was increased proportionately.

### Example 26

This experiment was run as described in Example 5 for the effect of pea protein on glucose consumption, biomass development and protein accumulation. The medium comprised 105 g/L date syrup and 6 g/L of urea. The inoculation ratio for all flasks was 5% v/v. Pea protein was tested at 0, 2, 4 and 6 g/L. The addition of pea protein was found to enhance glucose consumption, leading to higher biomass development and greater protein accumulation at all concentrations tested compared to the control lacking pea protein. Glucose consumption was estimated to have increased by 15-20% for all concentrations of pea protein tested. Regarding biomass development, each condition developed 31, 33, 33 and 33 g/L on average after ˜33 days (for 0, 2, 4 & 6 g/L pea protein respectively). Protein content, as measured by a Bradford assay, showed that protein had doubled between the control and all concentrations containing pea protein. In other words, the addition of pea protein potentiated glucose consumption, biomass development and protein accumulation, however, these results were observed at 2 g/L pea protein and further increases in pea protein did not result in further improvements in glucose consumption, biomass development and protein accumulation.

### Example 27: Production Conditions for P. Eryngii

Inoculum preparation. This step aims to propagate the fungal biomass from glycerol stocks to 2 L flasks with a working volume of 1.25 L. Pleurotus eryngii as described in Example 5 was the strain that was selected for this process. This step allowed the reactivation of the fungi and acclimated it to further growth in a media containing date syrup as a carbon source. This stage was run in duplicate.

2 separate 2 Liter Erlenmeyer flasks were filled with 1.21 L (Flask 1) and 0.04 L (Flask 2) of separate mediums shown in Table 11. The flasks were wrapped with a sterilizable bio-wrap which was wrapped with autoclave tape 5-6 times (the taped bio-wrap should be easily taken off and put back on the flask without losing shape) and sterilized in an autoclave that held the flasks at 120-121° C. for 90 minutes. The flasks were carefully transferred to a clean HEPA laminar flow-hood where they cooled for 18 hours. Flask 1 was added to Flask 2 and was subsequently inoculated with 2 cm² pieces of 60-day old PI Petri plate cultures of P. eryngii and placed on a shaker table at 120 rpm with a 1″ swing radius at 26° C. A microscope check was done to ensure the presence of mycelium and the culture was plated on LB media to ascertain the extent of any bacterial contamination. Inoculum is harvested when ready according to conditions provided in Example 5.

TABLE 11
Inoculum preparation
Parameters and Ranges

| Parameter | Operating Range | | Acceptable Range | |
|---|---|---|---|---|
| RO Water (Flask 1) | 1.21 | L | ±0.05 | L |
| RO Water (Flask 2) | 40 | mL | | |
| Date Syrup Charge (Flask | 37.5 | g | ±0 | g |
| 1) | | | | |
| Urea (Flask 2) | 2.5 | g | ±0 | g |
| Potassium Phosphate | 1.25 | g | ±0 | g |
| (Flask 2) | | | | |
| Magnesium Sulfate (Flask | 0.625 | g | ±0 | g |
| 2) | | | | |
| Fermentation Air Sparge | N/A | | N/A | |
| Flow Rate | | | | |
| Agitation Rate | 120 | RPM | N/A | |
| Media Sterilization | 123° | C. | ±2° | C. |
| Temperature | | | | |
| Media Sterilization Time | 0.5 | Hours | ±0.25 | Hours |
| Fermentation Temperature | 26° | C. | ±1° | C. |
| Fermentation Final pH | 5.5 | pH | ±0.2 | pH |

| | | |
|---|---|---|
| Fermentation Microscopy | No visible bacteria | — |
| Test | (100X) | |
| Petri film (24 h) | 0 | 0 |

S1 Fermentation: This fermentation was carried out in the S1 (25 L) bioreactor. The purpose of this step was to produce the inoculum for main fermenter (250 L). Media components (Table 11) were separately charged to the vessel and flasks to prevent Maillard reactions between the date syrup and protein sources., and then batch sterilized. The flasks were then transferred to a sterile 2.5 L vessel that is then transferred into the 25 L vessel also in a sterile manner. Next, the inoculum (5-7.5% inoculum ratio) was placed in a second, sterile 2.5 L vessel that was also transferred into S1. pH during fermentation was maintained between 5.0-5.50 through the addition of a sterilized citric acid and water solution in varying quantities based on the pH result provided every 12 hours. The fermentation was continued until glucose concentration, measured by YSI, was less than 2 g/L. This step is used to inoculate at 5-10% of the main fermentation. During this stage, samples of 100 mL were aseptically collected every 12 h to measure glucose concentration, pH and biomass. Immediately after sample collection, glucose was quantified as a representation of fungal growth. If the glucose content was lower than 2 g/L, the main fermentation was started by transferring S1 into it. Table 12: S1 Fermentation (27 L).

TABLE 12
S1 Fermentation (27 L)
Parameters and Ranges

| | | | Acceptable | |
|---|---|---|---|---|
| Parameter | Operating Range | | Range | |
| RO Water | 24.5 | L | ±0.5 | L |
| Date Syrup Charge | 750 | g | ±20 | g |
| (Vessel) | | | | |
| Pea Protein Charge (Flask) | 200 | g | ±5 | g |
| Yeast Extract (Flask) | 50 | g | ±5 | g |
| Potassium Phosphate | 25 | g | ±5 | g |
| (Flask) | | | | |
| Magnesium Sulfate (Flask) | 12 | g | ±5 | g |
| Citric Acid (Vessel) | 20 | g | ±5 | g |
| Antifoam (Flask) | 10 | g | ±5 | g |
| Fermentation Air Sparge | >0.75 | VVM | ±0.25 | VVM |
| Flow Rate | | | | |
| Media Sterilization | 123° | C. | ±2° | C. |
| Temperature | | | | |
| Media Sterilization Time | >1.50 | Hours | ±0.25 | Hours |
| Pre-Inoculation Sterilized | 5.25 | pH | ±0.25 | pH |
| Media pH | | | | |
| Pre-Inoculation Sterilized | 8 | g/L | ±2 | g/L |
| Media Glucose | | | | |
| Fermentation Time | Glucose | | ±0.5 | g/L |
| | Concentration <2.0 g/L | | | |
| Fermentation Temperature | 26° | C. | ±3° | C. |
| Fermentation Final pH | 5.5 | pH | ±1.0 | pH |

| Fermentation Microscopy | No visible, motile | — |
|---|---|---|
| Test | bacteria (100X) | |

| | | |
|---|---|---|
| Petri film (every 12 h) | 0 | 0 |

Main Fermentation: The main fermentation to produce biomass was carried out in the S2 (250 L) bioreactors. This step aimed to produce the final intermediate that consists of fungal biomass, and thus an increase in protein concentration. Media components were sterilized in a similar manner to S1 fermentation. Date Syrup, Citric Acid and RO water were added to S2, sterilized per the specifications in Table 12, then cooled to 26° C. The transfer line between S1 and S2 was sterilized, allowed to cool, then S1 (glucose concentration <2.0 g/L) was transferred to S2 (5-10% inoculum ratio). S1 was then cleaned, and the remaining media listed in Table 12 was added to it with 15 L of RO Water and sterilized per the specifications in Table 12 for S1. The transfer line from SI to S2 was sterilized again, and the media in S1 was then transferred to S2. The fermentation was sampled every 12 h during the fermentation. During the fermentation, pH is monitored and controlled (pH 5.0-5.5) by the addition of a citric acid and water solution in S1 the same way media was added.

The main fermentation was divided into two phases.

Phase 1. Initial Batch: In this first stage, the fungal biomass adapted to the new conditions and grew exponentially. The initial glucose was consumed, and then a fed-batch was carried out to continue the fungal growth. Phase 2, a fed-batch, started when the glucose concentration was below 4 g/L. See Table 13 for conditions.

Phase 2. Fed-batch. This Phase 2 allowed reaching of a higher fungal density. When the glucose concentration of the previous stage was lower than 4 g/L, the fed batch was initiated, adding the fed batch media in S1 to S2 in its entirety. Every 12 h, the reactor is sampled and, if the glucose concentration was lower than 4 g/L, an addition of sterilized concentrated media was done. The fed media were produced in S1 after inoculating S2 and cleaning the tank. The media components, as described in Table 14, were charged to the vessel, along with RO water, and then batch sterilized. A single fed-batch was added into S2. Again, pH was maintained between 5.0-5.5 after the fed-batch by the addition of sterilized citric acid and water solution. The fermentation was then allowed to reach a glucose concentration of 0.0 g/L, maintained for 24 hours, then heat treatment was initiated.

TABLE 13
S2 Main Fermentation (250 L)
Parameters and Ranges

| | | | Acceptable | |
|---|---|---|---|---|
| Parameter | Operating Range | | Range | |
| RO Water (Seed 2) | 190 | L | ±5% | |
| Date Syrup (Seed 2) | 7.00 | kg | ±0 | Kg |
| Pea Protein (Seed 1) | 2.000 | kg ( | ±0 | Kg |
| Urea (Seed 1) | 0.400 | kg | ±0 | Kg |
| Potassium Phosphate | 0.200 | kg | ±0 | Kg |
| (Seed 1) | | | | |
| Magnesium Sulfate (Seed | 0.400 | kg | ±0 | Kg |
| 1) | | | | |
| Antifoam (Seed 1) | .200 | kg | ±0 | Kg |
| Citric Acid (Seed 2) | 0.08 | kg | ±0 | Kg |
| Fermentation Air Sparge | 0.4 | VVM | ±0.5 | VVM |
| Flow Rate Start | | | | |
| Media Sterilization | 123° | C. | ±2° | C. |
| Temperature (S1, S2 | | | | |
| Media Sterilization Time | >1.50 | Hours | +0.25 | Hours |
| (S1, S2) | | | | |
| Pre-Inoculation Sterilized | 5-5.5 | pH | ±1.0 | pH |
| Media pH | | | | |
| Pre-Inoculation Sterilized | >8 | g/L | ±0.5 | g/L |
| Media Glucose | | | | |
| Agitation Rate | 100% Capacity | | ±0 | rpm |
| Fermentation Time | Once 25 L of Feed media | | ±3 | Hours |
| | is completed and, | | | |
| | Glucose is 0.0 g/L for 24 | | | |
| | hours or rheology of | | | |
| | media makes | | | |
| | fermentation inoperable. | | | |
| Fermentation Temperature | 26° | C. | ±4° | C. |
| Fermentation final pH | >6.0 | pH | ±0.5 | pH |

| | | |
|---|---|---|
| Fermentation Microscopy | No visible, motile | — |
| Test | bacteria (100X) | |
| Petri film (24 h) | 0 | 0 |

TABLE 14
Feed for the second stage of Main Fermentation
Parameters and Ranges

| | | | Acceptable | |
|---|---|---|---|---|
| Parameter | Operating Range | | Range | |
| RO Water | 25 | L | ±5 | L |
| Date Syrup Charge | 7.00 | KG | ±0 | kg |
| Pea Protein Charge | 2.000 | KG | ±0 | kg |
| Urea | 0.400 | | ±0 | kg |
| Antifoam | 0.500 | kg | ±10 | g |
| Potassium Phosphate | 0.200 | kg | ±10 | g |
| Magnesium Sulfate | 0.200 | | ±10 | g |
| Media Sterilization | 123° | C. | ±2° | C. |
| Temperature | | | | |
| Media Sterilization Time | >1.50 | Hours | +0.25 | hours |
| Pre-Inoculation Sterilized | 6 | pH | ±1.0 | pH |
| Media pH | | | | |
| Agitation Rate | Consistent with VVM | | Consistent with VVM | |
| Temperature | 23° | C. | ±3° | C. |

Heat Treatment: The purpose of this step was to complete the fermentation process by introducing steam and raising the temperature of the Main Fermenter to a temperature of 50° C. The Main Fermenter was held at 50° C. for at least 30 minutes to inactivate the biomass before being transferred into the filtration stage.

De-watering: the main fermentation post-heat treatment was then run through a de-watering separation process to have the biomass at a higher concentration before drying. The biomass is collected and kept for further drying. Specifically, a decanter was utilized which works by continuously feeding the main fermentation into the horizontal, rotating bowl decanter which is spinning at a high rotation per minute (RPM), leading to the separation of the solids from the liquid because of their density differences. The liquid and solids were separately collected, with the solids being further analyzed.

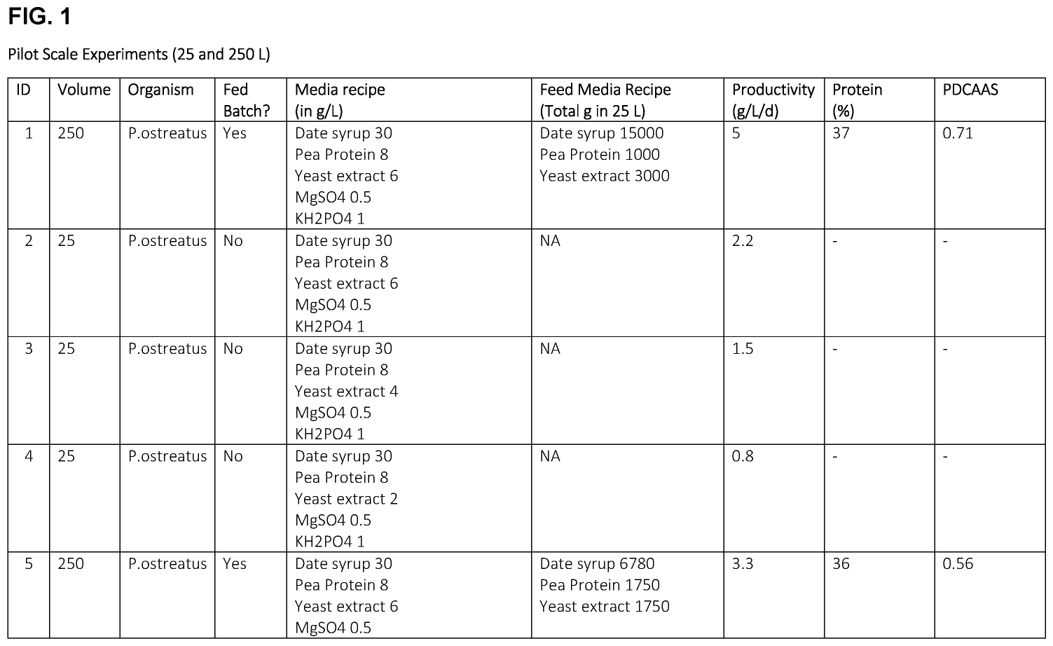

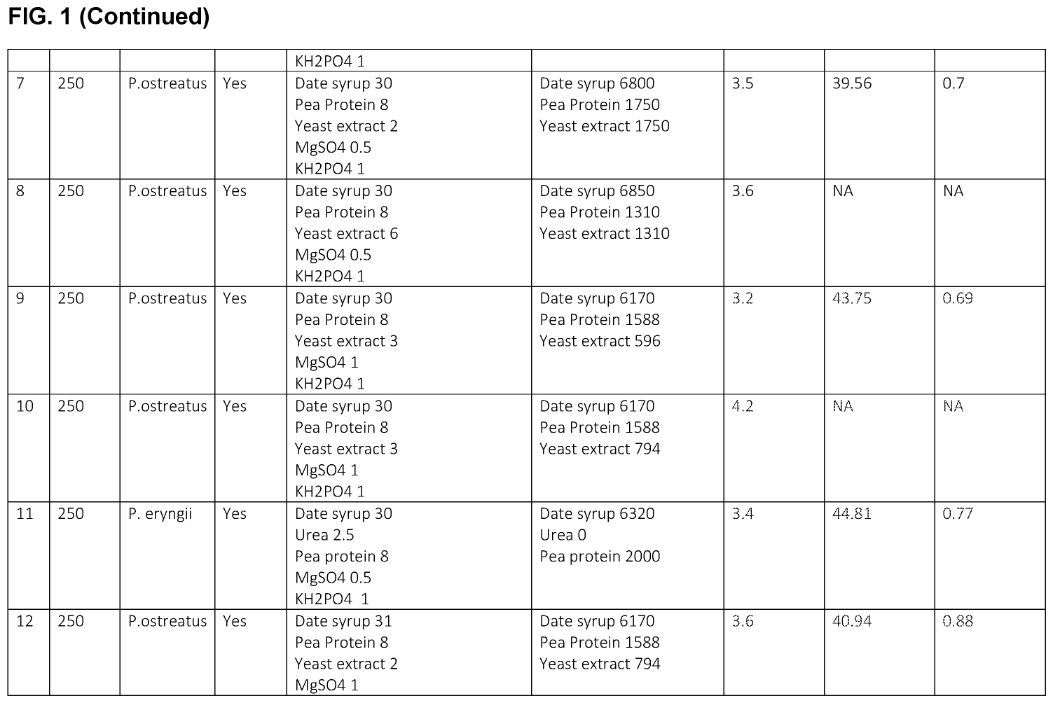

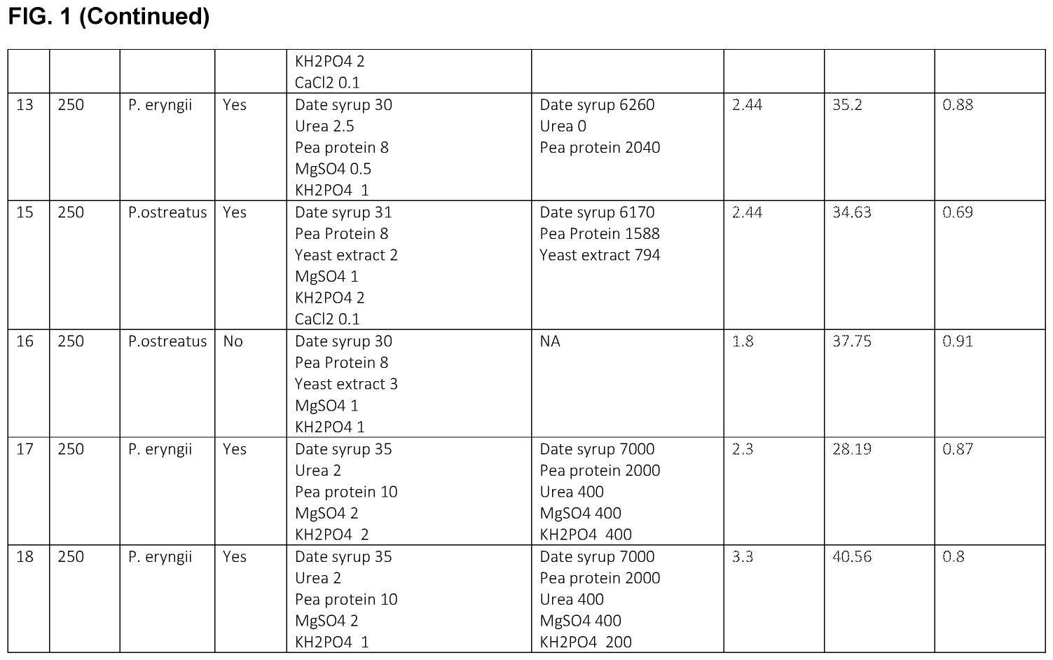

Yields from the fermentations runs according the conditions provided, according to the methods in this Example 26, can be seen in FIG. 1.

### Example 28

A “burger” made from the edible filamentous fungal biomass was made as follows. A biomass made by the process of Example 5 was lyophilized to approximately 7% solids and was used to create a burger as follows:

A texturized pea protein was hydrated with 1.75× water for 10-15 mins. Dry and ingredients were weighed and mixed together. In a stand mixer, the hydrated texturized pea protein was slowly mixed with the dry blend. The filamentous fungal biomass was slowly added and allowed to mix for 2 minutes. The remaining fat was added, and the mixture was mixed slowly until a cohesive mass formed/very first strands of gluten started showing. The mixture was chilled for about 1 hour, then formed into 113 g (4 oz) burger patties, and frozen.

Ingredients: all percentages by weight; filtered water, 8%; 7% solids filamentous fungus biomass, 10.6%; lyophilized filamentous fungus biomass (dry), 3.5%; filtered water (for texturized protein rehydration), 36%; textured protein, 20%, vital wheat gluten, 8%, TeamUp! Beef Lipid (Alianza Team®), 5%; methylcellulose, 2.7%; beet powder, 0.55%; flavors and seasonings, 5%.

Tasting Notes: Tasters noted that the texture of burger was ideal, closely resembled that of a ground beef patty and flavor was slight oat, fermented, with earthy finish.

### Example 29

A nondairy milk was made using the edible filamentous fungal biomass produced as in Example 5, which was lyophilized to approximately 7% solids, by the following method.

Ingredients were water, 91%, 7% solid filamentous fungal biomass, cane sugar 1.2%, gum, TIC Gum Blend Pro 181 AG (Acacia+Gellan), 0.2%, and fat mixture (AkoVeg™ 115-14, a blend of coconut oil, sunflower oil). The filamentous fungal biomass was mixed with water and allowed to hydrate for 5-10 minutes Gums were mixed with sugar and added to the water mixture, and allowed to mix for 5-10 minutes. The remaining ingredients were added and mixed until ready to process. Using a standard non-dairy milk processing apparatus, e.g., a MICROTHERMICS brand nondairy beverage steam injection processor, the following conditions were used to make the milk. Processing Conditions, Direct Steam Injection; HT: 4-5 sec; Homogenization: 2800 psi; Single Stage: 2300 psi; Second Stage: 500 psi. Tasters noted that the texture of the nondairy milk was smooth with few residual particles left on the palate.

### Example 30

Texturized protein. Biomass produced as discussed above in Example 5 is extruded to form a texturized protein. The extruded material has acceptable chewable characteristics and is almost identical in chewiness and firmness compared to a control produced with soy and has a clean, mild umami flavor and aroma.

### Processing Conditions:

A. Flour mix feed rate is 13 lbs at 17 lbs/min; preconditioner, water addition is in the range of 2.0 to 4.0 lbs/min; steam incorporation at 0.4 lb to 0.6 lbs/min; extruder; water addition ranged from 0.75 lbs to 1.0 lbs/min, extruder RPM is 350 to 400 rpm; extruder temperatures: zone 1, 155° F. to 165° F.; zone 2, 265° F. to 280° F.; zone 3, 285° F. to 295° F.; zone 4, 280° F. to 300° F.; pressures in the range of 800 to 1000 psi, dryer temperatures 250° F., 110° F.; product density 0.12 to 0.22 gm/cc; product moisture 5% to 7%.

Results: Successfully produce texturized protein. Improved texturization with pea fiber is achieved.

### Example 31

Raw mycelium (edible filamentous fungus biomass) is prepared as in Example 5 and is dewatered down to 70% water content using a screen and screw press. This material is shredded using a dough chopper into 1-50 mm chunks and is dried in an oven to 6% water content, then baked in an oven at 85° C. for 15 minutes for pasteurization. This dried material is then coated with an edible oil, flavorings, yeast extract, and fiber, and can be used in place of texturized vegetable protein to make burgers, or formed into “whole meat” products using art-known techniques such as adding binders and/or other ingredients and pressure, for example.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. As used herein, the term “about” indicates a reasonable range above and below a unit value, for instance +/−10% or +/1 unit, e.g. mm. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method to produce a composition comprising an edible filamentous fungal biomass comprising:
providing an aqueous media comprising a carbon source comprising an extract of dates; and a nitrogen source;
inoculating the media with a filamentous fungal culture, wherein the fungal culture comprises Pleurotus spp., and
culturing the filamentous fungal culture in a submerged fungal culture to produce the edible filamentous fungal biomass.

2. The method of claim 1, wherein the edible filamentous fungal biomass is grown to at least about 25 g/L (dry weight) with a productivity of at least 2.0 g/L/day (dry weight) during the culturing step.

3. The method of claim 1, wherein the extract of dates is date syrup having a ° Brix of at least about 70° Brix.

4. The method of claim 3, wherein the date syrup is initially present in the media at a concentration of between about 25 and 35 g/L, or between about 17° Brix and 24° Brix.

5. The method of claim 3, wherein the date syrup is initially present in the media at a concentration of between about 50 g/L and 110 g/L, or between about 40° Brix and 88° Brix.

6. The method of claim 1, wherein the nitrogen source in the media comprises urea, pea protein, yeast extract, or a combination thereof.

7. (canceled)

8. The method of claim 6, wherein the media comprises between about 2 g/L and 12 g/L pea protein and between about 1 g/L and 3 g/L urea.

9. The method of claim 1, wherein the culturing step comprises 7-12 days.

10. The method of claim 1, wherein the culturing step comprises a fed-batch culturing step.

11. The method of claim 10, wherein the fed-batch culturing step comprises feeding the culture with a media comprising glucose when measured glucose is below 4 g/L.

12. The method of claim 1, wherein the inoculum from the inoculation step is produced by a submerged fungal culturing step to produce an initial filamentous fungal biomass of at least 5 g/L (dry weight).

13. The method of claim 1, wherein the culturing step takes place in a bioreactor wherein the bioreactor has an impeller tip speed set during the culturing step of between 2 and 3 meters/second (m/s).

14. The method of claim 1, wherein when the filamentous fungus culture is selected from the group consisting of Pleurotus ostreatus, Pleurotus salmoneostramineus (Pleurotus djamor), Pleurotus eryngii, Pleurotus citrinopileatus, and combinations thereof.

15. The method of claim 14, wherein the filamentous fungus culture comprises Pleurotus eryngii.

16. The method of claim 1, wherein the aqueous media comprises date syrup of between about 17° Brix and 24° Brix, pea protein between about 5 g/L and 15 g/L, urea between about 1 g/L and 10 g/L, potassium phosphate between about 0.2 g/L and about 5 g/L, magnesium sulfate between about 0.1 g/L and 2 g/L, and thiamine between about 10 mg/L and 50 mg/L.

17. (canceled)

18. (canceled)

19. The method of claim 1, wherein the method further comprises the step of harvesting the edible filamentous fungus by dewatering.

20. (canceled)

21. The method of claim 1, further comprising the steps of dewatering the filamentous fungal biomass to produce a harvested filamentous fungal biomass comprising about 60-85% water and about 5-40% filamentous fungal biomass; pressing the harvested filamentous fungal biomass to produce a filamentous fungal biomass slab; shredding the filamentous fungal biomass slab to form shreds; and drying the shreds at about 50° C. to about 85° C. to form dried shreds.

22. (canceled)

23. (canceled)

24. A composition comprising an edible filamentous fungus, wherein the filamentous fungus is Pleurotus spp., which was cultured in a media comprising at least 20 g/L date extract and/or at least 16° Brix date syrup, and wherein the edible filamentous fungus was produced at a productivity of at least 20 g/L (dry weight).

25. The composition of claim 24, wherein when the filamentous fungus culture is selected from the group consisting of Pleurotus ostreatus, Pleurotus salmoneostramineus (Pleurotus djamor), Pleurotus eryngii, Pleurotus citrinopileatus, and combinations thereof.

26. The composition of claim 25, wherein the filamentous fungus culture comprises Pleurotus eryngii.

27. The composition of claim 24, wherein the date extract is date syrup having approximately between about 17° Brix and 24° Brix.

28. The composition of claim 24, wherein the media comprises a nitrogen source.

29. The composition of claim 28, wherein the nitrogen source comprises pea protein, urea, or combinations thereof.

30. The composition of claim 29, wherein the composition comprises between about 50% to approximately 80% filamentous fungal biomass.

31. (canceled)

32. (canceled)

33. The composition of claim 24, wherein the composition is a food composition selected from the group consisting of spreads, pastes, prewhipped toppings, custards, coatings, nut butters, frostings, cream filings, confectionery fillings, dairy alternative products, beverages and beverage bases, extruded and extruded/puffed products, meat imitations and extenders, baked goods and baking mixes, granola products, bar products, smoothies and juices, and soups and soup bases.
